(19) United States
(12) Patent Application Publication
(10) Pub. No.: US 2017/0287705 A1
(43) Pub. Date: Oct. 5, 2017

(54) Method for Cleaning Wafer, and Chemical Used in Such Cleaning Method

(71) Applicant: Central Glass Company, Limited, Ube-shi, Yamaguchi (JP)

(72) Inventors: Takashi SAIO, Toyonaka-shi, Osaka (JP); Yuzo OKUMURA, Ube-shi, Yamaguchi (JP); Yuki FUKUI, Ube-shi, Yamaguchi (JP); Soichi KUMON, Ube-shi, Yamaguchi (JP)

(73) Assignee: Central Glass Company ,Limited

(21) Appl. No.: 15/512,350

(22) PCT Filed: Sep. 11, 2015
(86) PCT No.: PCT/JP2015/075780

§ 371 Date: Mar. 17, 2017

(30) Foreign Application Priority Data
Sep. 18, 2014 (JP) 2014-190070
Aug. 28, 2015 (JP) 2015-169619

(51) Int. Cl. H01L 21/02; C07C 309/03; C07F 7/08

(52) U.S. Cl. CPC H01L 21/02087; H01L 21/0209; C07F 7/081; C07C 309/03

(57) ABSTRACT

Provided herein is a method for cleaning a wafer having a fine uneven surface pattern that at least partially contains a silicon element using a wafer cleaning device that includes a vinyl chloride resin as a liquid contacting member, the method including retaining a water-repellent protective film-forming chemical in at least a recessed portion of the uneven pattern to form a water-repellent protective film on a surface of the recessed portion, the water-repellent protective film-forming chemical containing:
a monoalkoxysilane represented by the following general formula [1],

(R¹)ₐSi(H)₃₋ₐ(OR²) [1];
a sulfonic acid represented by the following general formula [2],

R³—S(═O)₂OH [2]; and
a diluting solvent,
wherein the diluting solvent contains 80 to 100 mass % of alcohol with respect to the total 100 mass % of the diluting solvent.

### TECHNICAL FIELD

The present invention relates to a method for cleaning wafers with a predetermined chemical in cleaning wafers using a cleaning device that includes a vinyl chloride resin as a liquid contacting member.

### BACKGROUND ART

Wafer cleaning devices are available that use vinyl chloride resin for the member (liquid contacting member) that contacts a cleaning liquid or a processing liquid, as disclosed in PTL 1 to PTL 8. It is desirable that the cleaning liquid and the processing liquid used for such devices have properties that do not deteriorate the vinyl chloride resin. Examples of cleaning devices that include a vinyl chloride resin as a liquid contacting member include a wafer cleaning device in which vinyl chloride resin is used either in part or as a whole for members that contact the cleaning liquid or processing liquid in a cleaning vessel, and a wafer cleaning device in which vinyl chloride resin is used either in part or as a whole for a tank, pipes, joints, nozzles, and any other member that contact the cleaning liquid or processing liquid.

Semiconductor devices for networks and digital home electronics are required to meet the increasing demand for higher performance, higher functionality, and lower power consumption. A response to such demands is miniaturization of circuit patterns, and the increasing miniaturization has created a problem of falling circuit patterns. Production of a semiconductor device commonly involves a cleaning step intended to remove particles and metal impurities, and this step accounts for 30% to 40% of all semiconductor manufacturing steps. In a cleaning step, a high aspect ratio of patterns due to miniaturization of semiconductor devices causes the patterns to fall when the gas-liquid interface passes the patterns after cleaning or rinsing. This phenomenon is known as falling patterns. In order to prevent such falling patterns, it is often necessary to change the pattern design, and such an effort often leads to a low production yield. Therefore, this has created a need for a method that prevents falling patterns in a cleaning step.

It is known that forming a water-repellent protective film on a pattern surface is effective at preventing falling patterns. Because water repellency needs to be imparted without drying the pattern surface, the water-repellent protective film is formed using a water-repellent protective film-forming chemical that can make the pattern surface water repellent.

In PTL 9, the present applicant discloses a protective film-forming chemical for forming a water-repellent protective film on an uneven patterned surface of a wafer so that the cleaning step that often causes falling patterns can be improved without losing throughput in the production of a wafer having a fine uneven surface pattern that at least partially contains a silicon element. Specifically, a chemical is disclosed that forms a water-repellent protective film on at least a surface of a recessed portion of a fine uneven surface pattern of a wafer when cleaning a wafer having a fine uneven surface pattern that at least partially contains a silicon element. The water-repellent protective film-forming chemical includes a silicon compound A represented by the following general formula [A], and an acid for donating a proton to the silicon compound A and/or an acid for accepting an electron from the silicon compound A, and has a total moisture content of 5,000 ppm or less by mass in the starting raw material with respect to the total raw material amount. PTL 9 also discloses a wafer cleaning method using the chemical.

R¹ₐSi(H)_{b}(X)_{4-a-b} General Formula [A]

In formula [A], R¹ each independently represent at least one selected from a monovalent organic group including a hydrocarbon group having 1 to 18 carbon atoms, and a monovalent organic group including a fluoroalkyl chain of 1 to 8 carbon atoms, X each independently represent at least one selected from a halogen group, a monovalent organic group in which the element binding to Si is oxygen or nitrogen, and a nitrile group, a is an integer of 1 to 3, b is an integer of 0 to 2, and the sum of a and b is 3 or less.

### PRIOR ART LITERATURE

### Patent Literature

PTL 1: JP-A-05-259136
PTL 2: JP-A-07-245283
PTL 3: JP-A-10-189527
PTL 4: JP-A-10-229062
PTL 5: JP-A-11-283949
PTL 6: JP-A-2001-087725
PTL 7: JP-A-2008-098440
PTL 8: JP-A-2010-003739
PTL 9: JP-A-2012-033873

### DISCLOSURE OT THE INVENTION

### Technical Problem

The water-repellent protective film-forming chemical described in, for example, Example 4 of PTL 9 has a possibility of deteriorating vinyl chloride resin when used to clean a wafer having a fine uneven surface pattern that at least partially contains a silicon element using a wafer cleaning device that includes a vinyl chloride resin as a liquid contacting member.

It is accordingly an object of the present invention to provide a water-repellent protective film-forming chemical (hereinafter, also referred to as “protective film-forming chemical”, or simply “chemical”) that forms a water-repellent protective film (hereinafter, also referred to simply as “protective film”) on an uneven patterned surface of a wafer without deteriorating vinyl chloride resin when cleaning a wafer having a fine uneven surface pattern that at least partially contains a silicon element (hereinafter, such a wafer is also referred to simply as “wafer”) using a wafer cleaning device that includes a vinyl chloride resin as a liquid contacting member. The present invention is also intended to provide a wafer cleaning method using the chemical.

### Solution to Problem

The present invention is a method for cleaning a wafer having a fine uneven surface pattern that at least partially contains a silicon element using a wafer cleaning device that includes a vinyl chloride resin as a liquid contacting member,

the method including retaining a water-repellent protective film-forming chemical in at least a recessed portion of the uneven pattern to form a water-repellent protective film on a surface of the recessed portion, the water-repellent protective film-forming chemical containing:

a monoalkoxysilane represented by the following general formula [1];

a sulfonic acid represented by the following general formula [2]; and

a diluting solvent,

wherein the diluting solvent contains 80 to 100 mass % of alcohol with respect to the total 100 mass % of the diluting solvent,

(R¹)ₐSi(H)₃₋ₐ(OR²) [1],

wherein R¹ each independently represent at least one selected from monovalent hydrocarbon groups having 1 to 18 carbon atoms in which hydrogen elements may partially or totally be replaced with a fluorine element, R² represents a monovalent hydrocarbon group having 1 to 18 carbon atoms in which hydrogen elements may partially or totally be replaced with a fluorine element, and a is an integer of 1 to 3,

R³—S(═O)₂OH [2],

wherein R³ represents a group selected from the group containing a monovalent hydrocarbon group having 1 to 8 carbon atoms in which hydrogen elements may partially or totally be replaced with a fluorine element, and a hydroxyl group.

It is preferable that R³ of the sulfonic acid represented by the general formula [2] is a linear alkyl group having 1 to 8 carbon atoms in which hydrogen elements may partially or totally be replaced with a fluorine element.

It is preferable that the alcohol is a primary alcohol having 1 to 8 carbon atoms.

It is preferable that the monoalkoxysilane is at least one selected from the group containing monoalkoxysilanes represented by the following general formula [3]

R⁴—Si(CH₃)₂(OR⁵) [3],

wherein R⁴ represents a monovalent hydrocarbon group having 1 to 8 carbon atoms in which hydrogen elements may partially or totally be replaced with a fluorine element, and R⁵ represents a monovalent hydrocarbon group having 1 to 8 carbon atoms.

It is preferable that a concentration of the monoalkoxysilane in the water-repellent protective film-forming chemical is 0.5 to 35 mass %.

It is preferable that a concentration of the sulfonic acid in the water-repellent protective film-forming chemical is 0.1 to 30 mass %.

It is preferable that the water-repellent protective film-forming chemical is removed from the recessed portion by being dried after the water-repellent protective film is formed on the surface of the recessed portion with the water-repellent protective film-forming chemical retained in at least the recessed portion of the uneven pattern.

It is preferable that the water-repellent protective film-forming chemical in the recessed portion is replaced with a cleaning liquid different from the chemical, and the cleaning liquid is removed from the recessed portion by being dried after the water-repellent protective film is formed on the surface of the recessed portion with the water-repellent protective film-forming chemical retained in at least the recessed portion of the uneven pattern.

The water-repellent protective film may be removed by subjecting the dried wafer surface to at least one selected from the group containing a heat treatment, photo-irradiation, exposure to ozone, plasma irradiation, and corona discharge.

The present invention is a water-repellent protective film-forming chemical used when cleaning a wafer having a fine uneven surface pattern that at least partially contains a silicon element using a wafer cleaning device that includes a vinyl chloride resin as a liquid contacting member,

the water-repellent protective film-forming chemical comprising:
a monoalkoxysilane represented by the following general formula [1];
a sulfonic acid represented by the following general formula [2]; and
a diluting solvent,
wherein the diluting solvent contains 80 to 100 mass % of alcohol with respect to the total 100 mass % of the diluting solvent,

(R¹)ₐSi(H)₃₋ₐ(OR²) [1],

wherein R¹ each independently represent at least one selected from monovalent hydrocarbon groups having 1 to 18 carbon atoms in which hydrogen elements may partially or totally be replaced with a fluorine element, R² represents a monovalent hydrocarbon group having 1 to 18 carbon atoms in which hydrogen elements may partially or totally be replaced with a fluorine element, and a is an integer of 1 to 3,

R³—S(═O)₂OH [2],

wherein R³ represents a group selected from the group containing a monovalent hydrocarbon group having 1 to 8 carbon atoms in which hydrogen elements may partially or totally be replaced with a fluorine element, and a hydroxyl group.

It is preferable that R³ of the sulfonic acid represented by the general formula [2] is a linear alkyl group having 1 to 8 carbon atoms in which hydrogen elements may partially or totally be replaced with a fluorine element.

It is preferable that the alcohol is a primary alcohol having 1 to 8 carbon atoms.

It is preferable that the monoalkoxysilane is at least one selected from the group containing monoalkoxysilanes represented by the following general formula [3]

R⁴—Si(CH₃)₂(OR⁵) [3],

wherein R⁴ represents a monovalent hydrocarbon group having 1 to 8 carbon atoms in which hydrogen elements may partially or totally be replaced with a fluorine element, and R⁵ represents a monovalent hydrocarbon group having 1 to 8 carbon atoms.

It is preferable that a concentration of the monoalkoxysilane in the water-repellent protective film-forming chemical is 0.5 to 35 mass %.

It is preferable that a concentration of the sulfonic acid in the water-repellent protective film-forming chemical is 0.1 to 30 mass %.

### Advantageous Effects of the Invention

The water-repellent protective film-forming chemical of the present invention can form a water-repellent protective film on an uneven patterned surface of a wafer without deteriorating the vinyl chloride resin used for liquid contacting members in a wafer cleaning device. The protective film formed by the water-repellent protective film-forming chemical of the present invention has desirable water repellency, and lowers the capillary action on the uneven patterned surface of a wafer, and thereby prevents falling patterns. With the use of the chemical, the cleaning step in the production of a wafer having a fine uneven surface pattern can improve without lowering throughput. The water-repellent protective film-forming chemical of the present invention can thus improve the productivity of the wafer production producing a wafer having a fine uneven surface pattern.

The aspect ratio of wafer circuit patterns is expected to increase as the density continues to increase. The water-repellent protective film-forming chemical of the present invention is also applicable to cleaning of uneven patterns having an aspect ratio of, for example, 7 or more, and enables lowering the production cost of high density semiconductor devices. The chemical can be used without making large changes in existing devices such as in the liquid contacting member, and is applicable to production of a wide range of semiconductor devices.

### BRIEF EXPLANATION OF DRAWINGS

FIG. 1 is a schematic perspective view of a wafer 1 having a surface with a fine uneven pattern 2.

FIG. 2 is a diagram showing a partial cross section at a-a′ of FIG. 1.

FIG. 3 is a schematic view showing a state in which a protective film-forming chemical 8 is retained in a recessed portion 4 in a cleaning step.

FIG. 4 is a schematic view showing a state in which liquid is retained in a recessed portion 4 having a protective film formed thereon.

### DESCRIPTION OF EMBODIMENTS

(1) Water-Repellent Protective Film-Forming Chemical

A water-repellent protective film-forming chemical of the present invention includes:
a monoalkoxysilane represented by the following general formula [1];
a sulfonic acid represented by the following general formula [2]; and
a diluting solvent,
wherein the diluting solvent contains 80 to 100 mass % of alcohol with respect to the total 100 mass % of the diluting solvent,

(R¹)ₐSi(H)₃₋ₐ(OR²) [1],

wherein R¹ each independently represent at least one selected from monovalent hydrocarbon groups having 1 to 18 carbon atoms in which hydrogen elements may partially or totally be replaced with a fluorine element, R² represents a monovalent hydrocarbon group having 1 to 18 carbon atoms in which hydrogen elements may partially or totally be replaced with a fluorine element, and a is an integer of 1 to 3,

R³—S(═O)₂OH [2],

wherein R³ represents a group selected from the group containing a monovalent hydrocarbon group having 1 to 8 carbon atoms in which hydrogen elements may partially or totally be replaced with a fluorine element, and a hydroxyl group.

R¹ in the monoalkoxysilane is a water-repellent functional group. The alkoxy group (—OR² group) of the monoalkoxysilane reacts with the silanol group on wafer surface, and the monoalkoxysilane becomes immobilized thereon to form a water-repellent protective film on the wafer surface. By being used with the sulfonic acid, the monoalkoxysilane reacts with wafer surface at a higher rate, and provides a water repellency imparting effect.

Specific examples of the monoalkoxysilane include monomethoxysilanes such as (CH₃)₃SiOCH₃, C₂H₅Si(CH₃)₂OCH₃, (C₂H₅)₂Si(CH₃)OCH₃, (C₂H₅)₃SiOCH₃, C₃H₇Si(CH₃)₂OCH₃, (C₃H₇)₂Si(CH₃)OCH₃, (C₃H₇)₃SiOCH₃, C₄H₉Si(CH₃)₂OCH₃, (C₄H₉)₃SiOCH₃, C₅H₁₁Si(CH₃)₂OCH₃, C₆H₁₃Si(CH₃)₂OCH₃, C₇H₁₅Si(CH₃)₂OCH₃, C₈H₁₇Si(CH₃)₂OCH₃, C₉H₁₉Si(CH₃)₂OCH₃, C₁₀H₂₁Si(CH₃)₂OCH₃, C₁₁H₂₃Si(CH₃)₂OCH₃, C₁₂H₂₅Si(CH₃)₂OCH₃, C₁₃H₂₇Si(CH₃)₂OCH₃, C₁₄H₂₉Si(CH₃)₂OCH₃, C₁₅H₃₁Si(CH₃)₂OCH₃, C₁₆H₃₃Si(CH₃)₂OCH₃, C₁₇H₃₅Si(CH₃)₂OCH₃, C₁₈H₃₇Si(CH₃)₂OCH₃, (CH₃)₂Si(H)OCH₃, CH₃Si(H)₂OCH₃, (C₂H₅)₂Si(H)OCH₃, C₂H₅Si(H)₂OCH₃, C₂H₅Si(CH₃)(H)OCH₃, (C₃H₇)₂Si(H)OCH₃, CF₃CH₂CH₂Si(CH₃)₂OCH₃, C₂F₅CH₂CH₂Si(CH₃)₂OCH₃, C₃F₇CH₂CH₂Si(CH₃)₂OCH₃, C₄F₉CH₂CH₂Si(CH₃)₂OCH₃, C₅F₁₁CH₂CH₂Si(CH₃)₂OCH₃, C₆F₁₃CH₂CH₂Si(CH₃)₂OCH₃, C₇F₁₅CH₂CH₂Si(CH₃)₂OCH₃, C₈F₁₇CH₂CH₂Si(CH₃)₂OCH₃, and CF₃CH₂CH₂Si(CH₃)(H)OCH₃, and compounds in which the methyl moiety of the methoxy group of the methoxysilane is replaced with a methyl group in which the hydrogen elements are partially or totally replaced with fluorine elements, or compounds in which the methyl moiety of the methoxy group of the methoxysilane is replaced with a monovalent hydrocarbon group having 2 to 18 carbon atoms in which the hydrogen elements may partially or totally be replaced with fluorine elements.

From the viewpoint of water repellency imparting effect, and ease of maintaining water repellency after formation of the protective film, preferred as the monoalkoxysilane in the foregoing specific examples is one in which the number of R¹ groups, a, is 2 or 3, particularly preferably 3. The R² group of the monoalkoxysilane is preferably a monovalent hydrocarbon group having 1 to 18 carbon atoms, particularly preferably at least one selected from the group containing monoalkoxysilanes represented by the following general formula [3].

R⁴—Si(CH₃)₂(OR⁵) [3]

In formula [3], R⁴ represents a monovalent hydrocarbon group having 1 to 8 carbon atoms in hydrogen elements may partially or totally be replaced with a fluorine element, and R⁵ represents a monovalent hydrocarbon group having 1 to 8 carbon atoms.

Specific examples of the monoalkoxysilanes represented by the general formula [3] include alkyklimethylmonoalkoxysilanes such as (CH₃)₃SiOCH₃, C₂H₅Si(CH₃)₂OCH₃, C₃H₇Si(CH₃)₂OCH₃, C₄H₉Si(CH₃)₂OCH₃, C₅H₁₁Si(CH₃)₂OCH₃, C₆H₁₃Si(CH₃)₂OCH₃, C₇H₁₅Si(CH₃)₂OCH₃, C₈H₁₇Si(CH₃)₂OCH₃, CF₃CH₂CH₂Si(CH₃)₂OCH₃, C₂F₅CH₂CH₂Si(CH₃)₂OCH₃, C₃F₇CH₂CH₂Si(CH₃)₂OCH₃, C₄F₉CH₂CH₂Si(CH₃)₂OCH₃, C₅F₁₁CH₂CH₂Si(CH₃)₂OCH₃, and C₆F₁₃CH₂CH₂Si(CH₃)₂OCH₃, and compounds in which the methyl moiety of the methoxy group of the alkyldimethylmonoalkoxysilane is replaced with a monovalent hydrocarbon group having 2 to 8 carbon atoms. From the viewpoint of water repellency imparting effect, R⁴ is preferably a monovalent linear hydrocarbon group having 1 to 8 carbon atoms in which the hydrogen elements may partially or totally be replaced with fluorine elements, particularly preferably a methyl group. R⁵ is preferably an alkyl group having 1 to 8 carbon atoms in which the carbon atom binding to the oxygen atom is a primary carbon atom. Specific examples of such groups include compounds such as (CH₃)₃SiOCH₃, (CH₃)₃SiOC₂H₅, (CH₃)₃SiOCH₂CH₂CH₃, (CH₃)₃SiOCH₂CH₂CH₂CH₃, (CH₃)₃SiOCH₂CH(CH₃)₂, (CH₃)₃SiOCH₂CH₂CH₂CH₂CH₃, (CH₃)₃SiOCH₂CH₂CH(CH₃)₂, (CH₃)₃SiOCH₂CH₂CH₂CH₂CH₂CH₃, (CH₃)₃SiOCH₂CH₂CH₂CH(CH₃)₂, (CH₃)₃SiOCH₂CH₂CH₂CH₂CH₂CH₂CH₃, (CH₃)₃SiOCH₂CH₂CH₂CH₂CH(CH₃)₂, (CH₃)₃SiOCH₂CH₂CH₂CH₂CH₂CH₂CH₂CH₃, and (CH₃)₃SiOCH₂CH₂CH₂CH₂CH₂CH(CH₃)₂. Increasing the flash point of the monoalkoxysilane increases the flash point of the chemical, and the safety improves. From this viewpoint, R⁵ has preferably 3 to 8 carbon atoms, particularly preferably 4 to 8 carbon atoms.

The concentration of the monoalkoxysilane in the chemical is preferably 0.5 to 35 mass %. A concentration of 0.5 mass % or more is preferable because it makes it easier to exhibit the water repellency imparting effect. A concentration of 35 mass % or less is preferable because it makes vinyl chloride resin less likely to deteriorate. The concentration is more preferably 0.7 to 33 mass %, further preferably 1.0 to 31 mass %. The concentration of monoalkoxysilane in the chemical is the mass percent of monoalkoxysilane with respect to the total amount of the monoalkoxysilane represented by general formula [1], the sulfonic acid represented by general formula [2], and the diluting solvent.

The sulfonic acid promotes a reaction between the alkoxy group (—OR² group) of the monoalkoxysilane and the silanol group on wafer surface. Use of acids other than sulfonic acid may result in an insufficient water repellency imparting effect, or may cause deterioration of vinyl chloride resin.

Specific examples of the sulfonic acid include sulfuric acid, methanesulfonic acid, ethanesulfonic acid, butanesulfonic acid, octanesulfonic acid, benzenesulfonic acid, para-toluenesulfonic acid, trifluoromethanesulfonic acid, heptafluoropropanesulfonic acid, nonafluorobutanesulfonic acid, and tridecafluorohexanesulfonic acid. From the viewpoint of promoting the reaction (and, in turn, improving the water repellency imparting effect), R³ of the sulfonic acid represented by the general formula [2] is preferably a linear alkyl group having 1 to 8 carbon atoms in which the hydrogen elements may partially or totally be replaced with fluorine elements. R³ is further preferably a linear alkyl group having 1 to 8 carbon atoms, particularly preferably methanesulfonic acid.

The concentration of the sulfonic acid in the chemical is preferably 0.1 to 30 mass %. A concentration of 0.1 mass % or more is preferable because it helps exhibit the reaction promoting effect (and, in turn, the water repellency imparting effect). A concentration of 30 mass % or less is preferable because it makes the wafer surface less likely to corrode, or makes sulfonic acid less likely to remain as impurities on wafer. The concentration is more preferably 0.5 to 25 mass %, further preferably 1.0 to 20 mass %. The concentration of sulfonic acid in the chemical is the mass percent of sulfonic acid with respect to the total amount of the monoalkoxysilane represented by general formula [1], the sulfonic acid represented by general formula [2], and the diluting solvent.

The alcohol is used as a solvent to dissolve the monoalkoxysilane and the sulfonic acid. The alcohol may be one with more than one hydroxyl group. It is, however, preferable that the alcohol has only one hydroxyl group. The alcohol preferably has 1 to 8 carbon atoms because vinyl chloride resin becomes less likely to deteriorate when the alcohol has 8 or less carbon atoms. Specific examples of the alcohol include methanol, ethanol, 1-propanol, 2-propanol, 1-butanol, 2-butanol, isobutanol, tert-butanol, 1-pentanol, 2-pentanol, 3-pentanol, 2-methyl-1-butanol, 3-methyl-1-butanol, 2-methyl-2-butanol, 3-methyl-2-butanol, 1-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 3,3-dimethyl-1-butanol, 3,3-dimethyl-2-butanol, 2-ethyl-1-butanol, 1-heptanol, 2-heptanol, 3-heptanol, 4-heptanol, benzyl alcohol, 1-octanol, isooctanol, and 2-ethyl-1-hexanol. Preferred for water repellency imparting effect are primary alcohols such as methanol, ethanol, 1-propanol, 1-butanol, isobutanol, 1-pentanol, 2-methyl-1-butanol, 3-methyl-1-butanol, 1-hexanol, 2-methyl-1-pentanol, 3-methyl-1-pentanol, 4-methyl-1-pentanol, 2,2-dimethyl-1-butanol, 3,3-dimethyl-1-butanol, 2-ethyl-1-butanol, 1-heptanol, benzyl alcohol, 1-octanol, isooctanol, and 2-ethyl-1-hexanol. Increasing the flash point of the alcohol increases the flash point of the chemical, and the safety improves. From this viewpoint, the alcohol has preferably 3 to 8 carbon atoms, particularly preferably 4 to 8 carbon atoms.

The chemical of the present invention may contain an organic solvent other than the alcohol. However, from the viewpoint of preventing deterioration of vinyl chloride resin, the content of an organic solvent other than the alcohol is less than 20 mass % with respect to the total 100 mass % of the solvent. The organic solvent content is preferably less than 10 mass %, more preferably less than 5 mass %. Specifically, the alcohol is 80 to 100 mass %, preferably 90 to 100 mass %, more preferably 95 to 100 mass % of the total 100 mass % of the solvent.

Examples of the organic solvent other than alcohol include hydrocarbons, esters, ethers, ketones, halogen-containing solvents, sulfoxide solvents, lactone solvents, carbonate solvents, and derivatives of polyalcohol. Preferred are hydrocarbons, esters, ethers, ketones, halogen-containing solvents, and derivatives of polyalcohol. Hydrocarbons, ethers, and halogen-containing solvents are particularly preferred from the viewpoint of achieving a good balance between prevention of deterioration of vinyl chloride resin and water repellency imparting effect.

The monoalkoxysilane and the sulfonic acid contained in the chemical may be obtained by reaction. For example, these may be obtained through reaction of a silylation agent and an alcohol, as in the following formula [4].

(R¹)ₐSi(H)₃₋ₐ—OS(═O)₂—R³+R²OH→(R¹)ₐSi(H)₃₋ₐ—OR²+R³—S(═O)₂—OH [4]

In this reaction formula, R¹, R², and a are the same as in general formula [1], and R³ is the same as in general formula [2].

The sulfonic acid contained in the chemical may be obtained by reaction. For example, the sulfonic acid may be one obtained by reaction of an alcohol with sulfonic acid anhydrides (hereinafter, also referred to as “acid A”) such as methanesulfonic acid anhydrides, ethanesulfonic acid anhydrides, butanesulfonic acid anhydrides, octanesulfonic acid anhydrides, benzenesulfonic acid anhydrides, para-toluenesulfonic acid anhydrides, trifluoromethanesulfonic acid anhydrides, heptafluoropropanesulfonic acid anhydrides, nonafluorobutanesulfonic acid anhydrides, and tridecafluorohexanesulfonic acid anhydrides.

The total amount of moisture in the starting raw material of the chemical is preferably 5,000 ppm or less by mass of the total amount of the raw material. When the total moisture content is more than 5,000 ppm by mass, the monoalkoxysilane and the sulfonic acid become less effective, and it becomes difficult to form the protective film in a short time period. It is accordingly preferable that the total moisture content in the raw material of the chemical be as small as possible, specifically 1,000 ppm or less by mass, more preferably 500 ppm or less by mass. The moisture content should be reduced because excessive moisture contents tend to cause poor chemical storage stability, and is preferably 200 ppm or less by mass, further preferably 100 ppm or less by mass. The moisture content in the raw material of the chemical should be reduced for the reasons described above. However, the raw material of the chemical may contain 0.1 ppm or more by mass of moisture, provided that the content does not fall outside of the foregoing range. For these reasons, it is preferable that the monoalkoxysilane, the sulfonic acid, and the diluting solvent contained in the chemical have only a small moisture content.

The chemical in a liquid phase contains preferably at most 100 particles having a particle size larger than 0.2 μm per milliliter of the chemical, as measured by a particle measurement with a light scattering liquid particle detector. When the number of particles larger than 0.2 μm exceeds 100 per milliliter of the chemical, the particles may damage the pattern, and lower the yield and the reliability of the device, which is not preferable. On the other hand, it is preferable to contain at most 100 particles larger than 0.2 μm per milliliter of the chemical because it enables omitting or reducing the cleaning with solvent or water after the formation of the protective film. The number of particles larger than 0.2 μm should preferably be as small as possible. It is, however, possible to contain one or more particles larger than 0.2 μm per milliliter of the chemical, provided that the number is within the foregoing range. In the present invention, the liquid-phase particle measurement of the chemical or the processing liquid is performed with a commercially available light scattering liquid particle measurement device using a laser light source, and the particle size of particles means a light-scattering corresponding diameter relative to the PSL (polystyrene latex) standard particles.

Here, the particles include, for example, dust, dirt, organic solids, and inorganic solids contained as impurities in the raw material, and dust, dirt, organic solids, and inorganic solids that contaminate the chemical during its preparation. In other words, the particles include particles that remain in the chemical in the end without dissolving therein.

The content of Na, Mg, K, Ca, Mn, Fe, Cu, Li, Al, Cr, Ni, Zn, and Ag (metal impurities) in the chemical is preferably 0.1 ppb or less by mass for each element with respect to the total chemical amount. When the metal impurity content is more than 0.1 ppb by mass with respect to the total chemical amount, the junction leak current of the device may increase, and the device suffers from poor yield and poor reliability, which is not preferable. It is preferable to make the impurity content of each metal 0.1 ppb or less by mass with respect to the total chemical amount because it enables omitting or reducing the cleaning of wafer surface (protective film surface) with solvent or water after the formation of the protective film on wafer surface. For these reasons, the metal impurity content should preferably be reduced as much as possible. It is, however, possible to contain each element in an amount of 0.001 ppb or more by mass with respect to the total chemical amount, provided that the content is within the foregoing range.

(2) Water-Repellent Protective Film

In the present invention, the water-repellent protective film is a film that is formed on a wafer surface to reduce the wettability of the wafer surface. In other words, the water-repellent protective film is a film that imparts water repellency. As used herein, water repellency means to reduce the surface energy of article surface, and to thereby reduce, for example, hydrogen bonding, intermolecular force, and other such interaction (at the interface) between liquid, including water, and the article surface. Though the effect of the water-repellent protective film to reduce interaction is particularly strong against water, the water-repellent protective film also has the effect to reduce interaction against a mixture of water and liquids other than water, and against liquids other than water. By reducing interaction, the contact angle of liquid with the article surface can increase. The water-repellent protective film may be a film formed from the monoalkoxysilane, or a film containing a reaction product that contains monoalkoxysilane as a primary component.

(3) Wafer

The wafer may be one in which a film containing a silicon element such as silicon, silicon oxide, and silicon nitride is formed on a wafer surface, or one having an uneven pattern the surface of which at least partially contains a silicon element such as silicon, silicon oxide, and silicon nitride. The protective film also can be formed on a wafer configured from more than one component including at least a silicon element, specifically on a surface of components including a silicon element. The wafer configured from more than one component may be a wafer in which components including a silicon element such as silicon, silicon oxide, and silicon nitride is formed on a wafer surface, or a wafer having an uneven pattern that at least partially contains a silicon element such as silicon, silicon oxide, and silicon nitride. The chemical can form the protective film on a surface in a part of the uneven pattern containing a silicon element.

A wafer having a fine uneven surface pattern is typically obtained as follows. First, a resist is applied to a flat and smooth wafer surface, and exposed through a resist mask. The exposed or unexposed resist is then etched away to produce a resist having a desired uneven pattern. A resist having an uneven pattern also can be obtained by pressing a patterned mold against a resist. This is followed by wafer etching. Here, the etching selectively etches a wafer surface corresponding to recessed portions of the resist pattern. Finally, the resist is released to obtain a wafer having a fine uneven pattern.

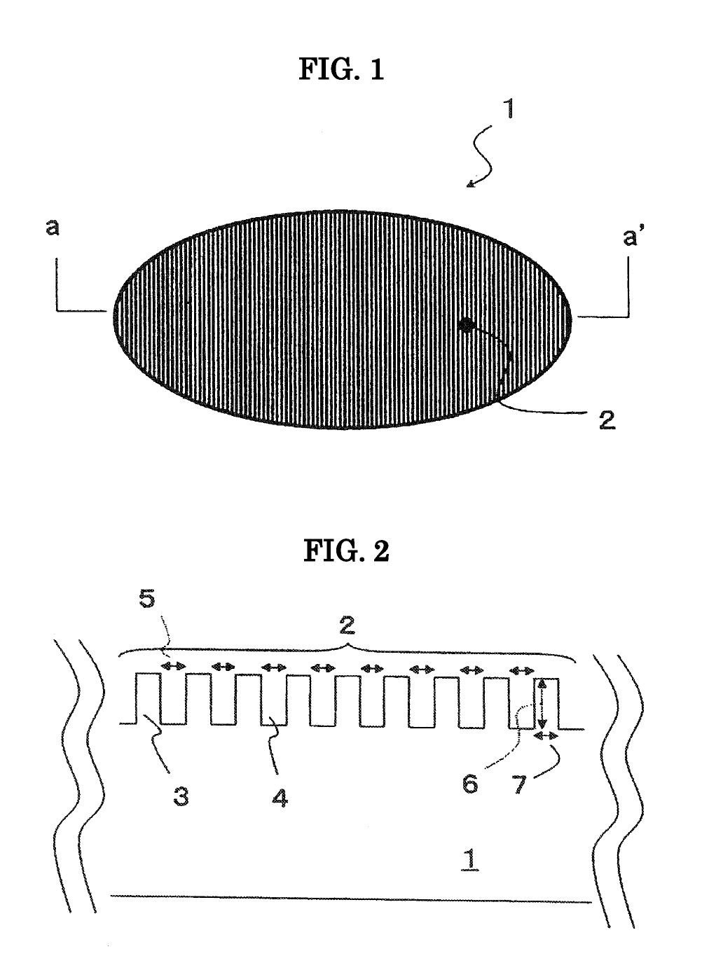

After forming a fine uneven pattern in the wafer surface, the surface is cleaned with a water-based cleaning liquid. The water-based cleaning liquid is then removed by, for example, drying. Here, falling patterns tend to occur when the raised portion has a large aspect ratio with a narrow recessed portion. The uneven pattern is defined as shown in FIGS. 1 and 2. FIG. 1 shows a schematic perspective view of a wafer 1 having a surface with a fine uneven pattern 2. FIG. 2 is a diagram showing a partial cross section at a-a′ of FIG. 1. The recessed portion has a width 5 defined by the distance between the adjacent raised portions 3, as shown in FIG. 2, and the aspect ratio of the raised portion is a ratio obtained by dividing the height 6 of the raised portion by the width 7 of the raised portion. Falling patterns tend to occur in the clean step when the recessed portion has a width of 70 nm or less, particularly 45 nm or less, or when the aspect ratio is 4 or more, particularly 6 or more.

(4) Wafer Cleaning Method

The wafer having a fine uneven surface pattern after etching may be cleaned with a water-based cleaning liquid to remove etching residues or the like before being cleaned by the cleaning method of the present invention, or the cleaned wafer may be further cleaned by replacing the water-based cleaning liquid retained in the recessed portion with a cleaning liquid (hereinafter, “cleaning liquid A”) different from the water-based cleaning liquid.

Examples of the water-based cleaning liquid include water, and an aqueous solution (a water content of, for example, 10 mass % or more) as a mixture of water with at least one of an organic solvent, hydrogen peroxide, ozone, an acid, an alkali, and a surfactant.

The cleaning liquid A refers to an organic solvent, a mixture of an organic solvent and the water-based cleaning liquid, or a cleaning liquid as a mixture of these with at least one of an acid, an alkali and a surfactant.

In the present invention, the method of cleaning the wafer is not particularly limited, as long as a cleaning device is used that can retain the chemical and the cleaning liquid in at least the recessed portion of the uneven pattern of the wafer. The wafer cleaning method may be, for example, a method in which wafers are cleaned one by one using a spin cleaning device that supplies the liquid near the center of rotation of a spinning wafer held substantially horizontally, or a batch method using a cleaning device that cleans a plurality of wafers clipped in a cleaning vessel. The chemical or the cleaning liquid supplied to at least the recessed portion of the uneven pattern of the wafer may have any form, including, for example, a liquid, and a steam, provided that the chemical and the cleaning liquid are retained in liquid form in the recessed portion.

Examples of the organic solvent as a preferred example of the cleaning liquid A include hydrocarbons, esters, ethers, ketones, halogen-containing solvents, sulfoxide solvents, lactone solvents, carbonate solvents, alcohols, derivatives of polyalcohol, and nitrogen-containing solvents.

The protective film-forming chemical of the present invention is used by replacing the water-based cleaning liquid or the cleaning liquid A. The chemical that has replaced these liquids may be replaced with a cleaning liquid (hereinafter, “cleaning liquid B”) different from the chemical.

The water-based cleaning liquid or the cleaning liquid A used to clean the wafer in the manner described above is replaced with the protective film-forming chemical, and the protective film is formed on the surface of at least the recessed portion of the uneven pattern while the chemical is retained in at least the recessed portion of the uneven pattern. It is not necessarily required to continuously or uniformly form the protective film of the present invention. It is, however, preferable to form a uniform, continuous protective film because such a film can impart more desirable water repellency.

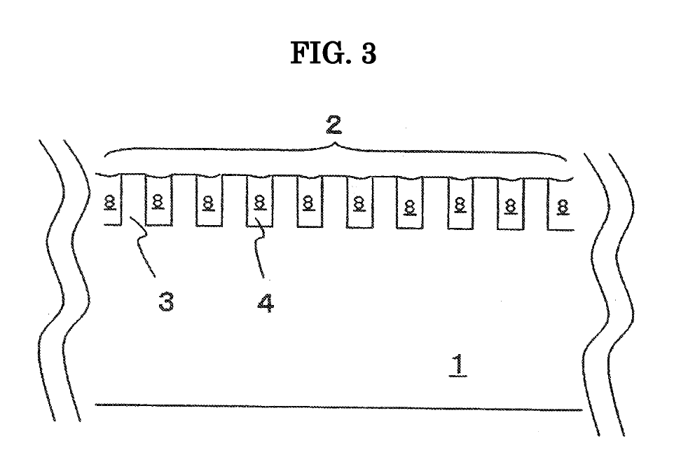

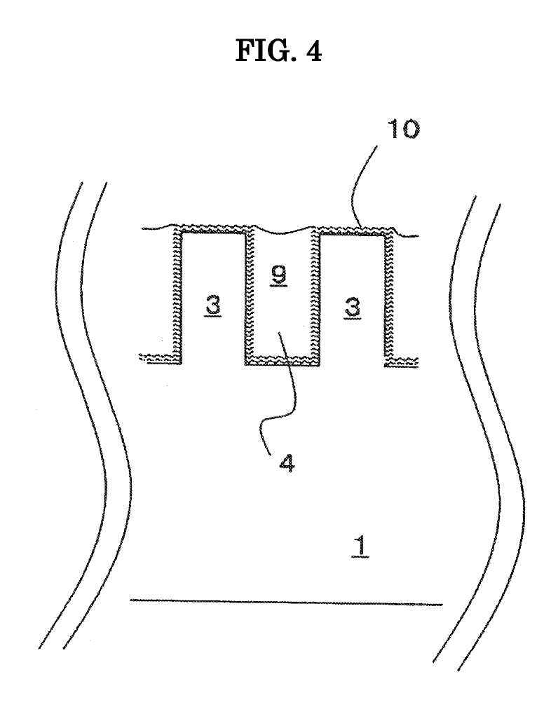

FIG. 3 is a schematic view showing a state in which a protective film-forming chemical 8 is retained in the recessed portion 4. FIG. 3 schematically shows a part of the wafer in a cross section at a-a′ of FIG. 1. The protective film is formed on the surface of the recessed portion 4 to make the surface water repellent.

The protective film-forming chemical can more quickly form the protective film at elevated temperatures. A more uniform protective film can be formed at a temperature of 10° C. or more and less than the boiling point of the chemical. Particularly, it is preferable to retain the chemical in a temperature range between 15° C. or more and at least 10° C. below the boiling point of the chemical. Preferably, the chemical is held at these temperatures also when the chemical is retained in at least the recessed portion of the uneven pattern. The boiling point of the chemical is the boiling point of the most abundant component of the protective film-forming chemical in terms of a mass ratio.

After forming the protective film in the manner described above, the chemical remaining in at least the recessed portion of the uneven pattern may be replaced with cleaning liquid B before starting a drying step. Examples of the cleaning liquid B include water-based cleaning liquids, organic solvents, a mixture of a water-based cleaning liquid and an organic solvent, a mixture of these with at least one of an acid, an alkali, and a surfactant, and a mixture of these with the protective film-forming chemical. From the viewpoint of removing particles and metal impurities, the cleaning liquid B is preferably water, an organic solvent, or a mixture of water and an organic solvent.

Examples of the organic solvent as a preferred example of the cleaning liquid B include hydrocarbons, esters, ethers, ketones, halogen-containing solvents, sulfoxide solvents, alcohols, derivatives of polyalcohol, and nitrogen-containing solvents.

When an organic solvent is used as the cleaning liquid B, the protective film formed on wafer surface by the chemical of the present invention is less likely to suffer from poor water repellency after washing with the cleaning liquid B.

FIG. 4 is a schematic view showing a state in which liquid is retained in the recessed portion 4 rendered water repellent by the protective film-forming chemical. FIG. 4 schematically shows a part of the wafer in a cross section at a-a′ of FIG. 1. A protective film 10 is formed on the surface of the uneven pattern to make the surface water repellent. The protective film 10 remains on the wafer surface even after liquid 9 is removed from the uneven pattern.

With the protective film 10 formed by the protective film-forming chemical on the surface of at least the recessed portion of the uneven pattern of the wafer, the contact angle of the liquid retained on the surface should be 50 to 130° in the case of water. This is preferable because it makes falling patterns less likely to occur. Water repellency becomes more desirable as the contact angle increases, and the contact angle is further preferably 60 to 130°, particularly preferably 65 to 130°. A reduction of contact angle before and after cleaning with the cleaning liquid B (contact angle before cleaning with cleaning liquid B−contact angle after cleaning with cleaning liquid B) is preferably 10° or less.

The liquid retained in the recessed portion 4 having the protective film formed thereon with the chemical is dried and removed from the uneven pattern. Here, the liquid retained in the recessed portion may be the chemical, the cleaning liquid B, or a mixture of these. The mixture contains the components of the protective film-forming chemical in lower concentrations than in the chemical, and may be a liquid from a transition phase of the chemical being replaced with the cleaning liquid B, or a mixture obtained in advance by mixing the components with the cleaning liquid B. From the viewpoint of wafer cleanness, it is preferable to use water, an organic solvent, or a mixture of water and an organic solvent. After removing the liquid from the uneven patterned surface, the cleaning liquid B may be retained on the uneven patterned surface, and dried.

When cleaning the wafer with cleaning liquid B after forming the protective film, the cleaning time, i.e., the retention time of the cleaning liquid B, is preferably 10 seconds or more, more preferably 20 seconds or more from the viewpoint of removing particles and impurities from the uneven patterned surface. In terms of the water repellency maintaining effect of the protective film formed on the uneven patterned surface, the wafer surface becomes more likely to remain water repellent even after the cleaning when an organic solvent is used as cleaning liquid B. The cleaning time is preferably at most 15 minutes because an excessively long cleaning time results in poor productivity.

The liquid retained in the uneven pattern is removed by drying. Preferably, the liquid is dried using known drying methods such as spin drying, IPA (2-propanol) steam drying, Marangoni drying, heat drying, hot-air drying, air drying, and vacuum drying.

The protective film 10 may be removed after the drying. When removing the water-repellent protective film, it is effective to cut the C—C bond and the C—F bond in the water-repellent protective film. The method is not particularly limited, as long as the bonds can be cut. For example, the water-repellent protective film may be removed by irradiating the wafer surface with light, heating the wafer, exposing the wafer to ozone, irradiating the wafer surface with a plasma, or subjecting the wafer surface to corona discharge.

When removing the protective film 10 by photo-irradiation, it is preferable to use ultraviolet light of wavelengths shorter than 340 nm and 240 nm, which correspond to the bond energies 83 kcal/mol and 116 kcal/mol of the C—C bond and C—F bond, respectively, of the protective film 10. The light source may be, for example, a metal halide lamp, a low-pressure mercury lamp, a high-pressure mercury lamp, an excimer lamp, or a carbon arc. In the case of a metal halide lamp, the ultraviolet irradiation intensity is preferably 100 mW/cm² or more, particularly preferably 200 mW/cm² or more as measured with, for example, an illuminometer (Irradiation Intensity Meter UM-10, and Photoreceiver UM-360 from Konica Minolta Sensing [peak sensitivity wavelength: 365 nm, measurement wavelength range: 310 to 400 nm]). It takes a long time to remove the protective film 10 when the irradiation intensity is less than 100 mW/cm². A low-pressure mercury lamp is preferable because it enables irradiation of ultraviolet rays of shorter wavelength, and allows the protective film 10 to be quickly removed even with a low irradiation intensity.

When removing the protective film 10 by photo-irradiation, it is particularly preferable to generate ozone at the time when the constituent components of the protective film 10 are decomposed by ultraviolet light, and to oxidatively evaporate the constituent components of the protective film 10 with the ozone because this reduces the process time. The light source may be, for example, a low-pressure mercury lamp, or an excimer lamp. The wafer may be heated while being irradiated with light.

When heating the wafer, it is preferable that the wafer be heated at 400 to 1,000° C., preferably 500 to 900° C. The heating time is 10 seconds to 60 minutes, preferably 30 seconds to 10 minutes. This step may accompany, for example, exposure to ozone, plasma irradiation, or corona discharge. Light may be applied while heating the wafer.

The protective film 10 may be heated and removed by, for example, contacting the wafer to a heat source, or placing the wafer in a heated atmosphere such as in a heating furnace. The latter method whereby a wafer is placed in a heated atmosphere takes less effort to uniformly apply energy to the wafer surface for the removal of the protective film 10 even when a plurality of wafers is processed, and is industrially advantageous in terms of processability including high ease of operation and a short process time.

When exposing the wafer to ozone, it is preferable that the ozone supplied to wafer surface is ozone generated by UV irradiation with a low-pressure mercury lamp or the like, or ozone generated by low-temperature discharge under high voltage. The wafer may be irradiated with light or heated while being exposed to ozone.

The protective film on wafer surface can be efficiently removed by performing the photo-irradiation, heating, exposure to ozone, plasma irradiation, and corona discharge in combination.

### EXAMPLES

The embodiment of the present invention is more specifically disclosed in the Examples below. It is to be noted that the present invention is not limited to the following Examples.

The technique that forms an uneven surface pattern on a wafer, and the technique that uses a different cleaning liquid to replace the cleaning liquid retained in at least the recessed portion of the uneven pattern have been examined in the literature, and these are already established. The present invention, therefore, evaluated the water repellency imparting effect of the protective film-forming chemical, and the resistance of vinyl chloride resin to the chemical. In the following Examples, the liquid brought into contact with wafer surface for the evaluation of contact angle is water, a typical water-based cleaning liquid.

In the case of a wafer having an uneven surface pattern, it is not possible to accurately evaluate the contact angle of the protective film 10 itself formed on the uneven patterned surface.

A contact angle of a water droplet is evaluated by dropping several microliters of water droplet onto a sample (base material) surface, and measuring the angle created between the water droplet and the base material surface, as described in JIS R 3257, Method for Testing Wettability of Glass Substrate Surface. However, a wafer having a pattern produces a very large contact angle. This is because of the Wenzel effect or the Cassie effect, whereby the surface shape (roughness) of the base material affects the contact angle, and increases the apparent contact angle of a water droplet.

In the following Examples, the chemical was applied to a wafer having a smooth surface, and the protective film was formed on the wafer surface. Evaluations were made by regarding the protective film as being formed on a wafer having an uneven surface pattern. In Examples, a wafer with a SiO₂ film having a SiO₂ layer formed on a smooth surface of a silicon wafer was used as the wafer having a smooth surface.

Details are as follows. The following describes the evaluation method, preparation of a protective film-forming chemical, a wafer cleaning method using the protective film-forming chemical, and results of evaluations after the formation of a protective film on a wafer.

[Evaluation Method]

Wafers having a protective film were evaluated using the following methods (A) to (C).

(A) Contact Angle Evaluation of Protective Film Formed on Wafer Surface

About 2 μl of purified water was placed on a wafer surface having a protective film, and the angle (contact angle) created between a water droplet and the wafer surface was measured with a contact angle meter (Model CA-X from Kyowa Interface Science Co., Ltd.).

(B) Reduction of Contact Angle Upon Contact with Water

The wafer having the protective film was dipped in 60° C. hot water for 10 minutes, and a reduction of contact angle was evaluated. Smaller reductions of contact angle mean that the contact angle is less likely to decrease in the cleaning after the formation of the protective film. A reduction of 10° or less is particularly preferred.

(C) Resistance of Vinyl Chloride Resin to Protective Film-Forming Chemical

In the Examples of the present invention, a vinyl chloride resin was dipped in the protective film-forming chemical, and evaluated for the presence or absence of deterioration, instead of evaluating the presence or absence of deterioration of a vinyl chloride resin included as a liquid contacting member in a wafer cleaning device by actually using the device to clean a wafer. Specifically, a vinyl chloride resin (with a gloss surface) was dipped in the protective film-forming chemical, and maintained at 40° C. for 4 weeks. The vinyl chloride resin was then visually inspected for any deterioration, and the presence or absence of deterioration, including discoloration and swelling, was confirmed. The vinyl chloride resin was considered acceptable when there was no deterioration, and unacceptable when deterioration was present.

### Example 1

(1) Preparation of Protective Film-Forming Chemical

A protective film-forming chemical was obtained by mixing 20 g of trimethylhexoxysilane [(CH₃)₃Si—OC₆H₁₃] as the raw material monoalkoxysilane, 10 g of methanesulfonic acid [CH₃S(═O)₂OH] as the sulfonic acid, and 70 g of 1-hexanol [CH₃CH₂CH₂CH₂CH₂CH₂—OH: nHA] as the diluting solvent.

(2) Cleaning of Silicon Wafer

A smooth silicon wafer with a thermally-oxidized film (a Si wafer with a 1-μm layer of a thermally-oxidized film formed on wafer surface) was clipped in a 1 mass % hydrofluoric acid aqueous solution for 10 minutes at room temperature. The silicon wafer was then dipped in purified water for 1 minute at room temperature, and in 2-propanol (iPA) for 1 minute at room temperature.

(3) Surface Treatment of Silicon Wafer Surface with Protective Film-Forming Chemical

The cleaned silicon wafer was dipped in the protective film-forming chemical prepared according to Preparation of Protective Film-Forming Chemical in Section (1) above. Here, the silicon wafer was kept in the chemical for 2 minutes at room temperature. The silicon wafer was then dipped in iPA for 1 minute at room temperature, and in purified water for 1 minute at room temperature. The silicon wafer was taken out of the purified water, and air was blown to remove purified water from the surface.

The wafer was evaluated according to the procedures described in (A) to (C) above. As shown in Table 1, an initial contact angle of less than 10° before surface treatment increased to 78° after the surface treatment, demonstrating the water repellency imparting effect. There was no reduction (0°) in contact angle, and the water repellency was desirably maintained. The vinyl chloride resin did not deteriorate even after being stored for 4 weeks at 40° C., and the resistance was desirable.

TABLE 1

| | Protective film-forming chemical | | | | | | | | | |
|---|---|---|---|---|---|---|---|---|---|---|
| | Starting raw materials | | | | | | | | | |
| | | | | | Diluting solvent | | Evaluation results | | | |
| | | | | | | Alcohol | | | Reduction | |
| | Monoalkoxysilane | | Sulfonic acid | | | concen- | | Contact | of contact | |
| | | Concen- | | Concen- | | tration | Initial | angle after | angle after | Resistance |
| | | tration | | tration | | in diluting | contact | surface | dipping in | of vinyl |
| | | [mass | | [mass | | slovent | angle | treatment | hot water | chloride |
| | Type | %] | Type | %] | Type | [mass %] | [°] | [°] | [°] | resin |
| Example 1 | (CH₃)₃Si—O₆H₁₃ | 20 | CH₃S(═O)₂OH | 10 | nHA | 100 | <10 | 78 | 0 | Acceptable |
| Example 2 | | 10 | | 10 | | | <10 | 75 | 0 | Acceptable |
| Example 3 | | 5 | | 10 | | | <10 | 70 | 0 | Acceptable |
| Example 4 | | 1 | | 10 | | | <10 | 66 | 0 | Acceptable |
| Example 5 | | 0.5 | | 10 | | | <10 | 63 | 0 | Acceptable |
| Example 6 | (CH₃)₃Si—O₆H₁₃ | 20 | CH₃S(═O)₂OH | 5 | nHA | 100 | <10 | 73 | 0 | Acceptable |
| Example 7 | | 10 | | 5 | | | <10 | 70 | 0 | Acceptable |
| Example 8 | | 5 | | 5 | | | <10 | 65 | 0 | Acceptable |
| Example 9 | | 1 | | 5 | | | <10 | 61 | 0 | Acceptable |
| Example 10 | | 0.5 | | 5 | | | <10 | 57 | 0 | Acceptable |
| Example 1 | (CH₃)₃Si—O₆H₁₃ | 20 | CH₃S(═O)₂OH | 10 | nHA | 100 | <10 | 78 | 0 | Acceptable |
| Example 6 | | 20 | | 5 | | | <10 | 73 | 0 | Acceptable |
| Example 11 | | 20 | | 1 | | | <10 | 70 | 0 | Acceptable |
| Example 12 | | 20 | | 0.5 | | | <10 | 68 | 0 | Acceptable |
| Example 13 | | 20 | | 0.1 | | | <10 | 64 | 0 | Acceptable |
| Example 1 | (CH₃)₃Si—O₆H₁₃ | 20 | CH₃S(═O)₂OH | 10 | nHA | 100 | <10 | 78 | 0 | Acceptable |
| Example 14 | | | | | nBA | 100 | <10 | 78 | 0 | Acceptable |
| Example 15 | | | | | nPA | 100 | <10 | 76 | 0 | Acceptable |
| Example 16 | | | | | EA | 100 | <10 | 75 | 0 | Acceptable |
| Example 17 | | | | | nPA/PGMEA-95 | 95 | <10 | 76 | 0 | Acceptable |
| Example 18 | | | | | iBA | 100 | <10 | 78 | 0 | Acceptable |
| Example 19 | | | | | iPA | 100 | <10 | 64 | 0 | Acceptable |
| Example 20 | | | | | 2BA | 100 | <10 | 64 | 0 | Acceptable |
| Example 21 | | | | | tBA | 100 | <10 | 52 | 0 | Acceptable |

### Examples 2 to 21

The wafer was subjected to the same surface treatment, and evaluated in the same manner as in Example 1, except that some conditions, including the concentrations of monoalkoxysilane and sulfonic acid, and the type of diluting solvent were varied from those used in Example 1. The results are presented in Table 1. In the table, “nBA” means 1-butanol, “nPA” means 1-propanol, “EA” means ethanol, “nPA/PGMEA-95” means a mixed solvent of nPA and PGMEA (propylene glycol monomethyl ether acetate) in a mass ratio of 95:5, “iPA” means 2-propanol, “iBA” means isobutanol, “2BA” means 2-butanol, and “tBA” means tert-butanol.

In all of Examples 2 to 21, an initial contact angle of less than 10° before surface treatment increased after the surface treatment, demonstrating the water repellency imparting effect. There was only a small reduction of contact angle, and the water repellency was desirably maintained. The vinyl chloride resin did not deteriorate even after being stored for 4 weeks at 40° C., and the resistance was desirable.

### Comparative Examples 1 to 210

As shown in Tables 2 to 6, the wafer was subjected to the same surface treatment, and evaluated in the same manner as in Example 1, except that some conditions, including the type and the concentration of alkoxysilane, the type and the concentration of acid, and the type of diluting solvent were varied from those used in Example 1.

Comparative Examples 1 to 3, 22 to 24, 43 to 45, 64 to 66, 85 to 87, 106 to 108, 127 to 129, 148 to 150, and 169 to 171 represent examples using a protective film-forming chemical that did not contain sulfonic acid. In these comparative examples, the contact angle after the surface treatment remained low at less than 10°, and the water repellency imparting effect was not observed.

Comparative Examples 4 to 12, 25 to 33, 46 to 54, 67 to 75, 88 to 96, 109 to 117, 130 to 138, 151 to 159, and 172 to 180 represent examples in which a protective film-forming chemical was used that contained acetic acid [CH₃C(═O)OH] instead of methanesulfonic acid. In these comparative examples, the contact angle after the surface treatment remained low at less than 10°, and the water repellency imparting effect was not observed.

Comparative Examples 13 to 21, 34 to 42, 55 to 63, 76 to 84, 97 to 105, 118 to 126, 139 to 147, 160 to 168, and 181 to 189 represent examples in which a protective film-forming chemical was used that contained methyltrimethoxysilane [(CH₃)Si(OCH₃)₃] instead of trimethylhexoxysilane. The water repellency imparting effect was insufficient in these comparative examples.

Comparative Examples 190 to 198 represent examples in which nPA/PGMEA-50 (a mixed solvent of nPA and PGMEA in a mass ratio of 50:50) was used as the diluting solvent. The vinyl chloride resin deteriorated (swelling was observed) after being stored for 4 weeks at 40° C., and the resistance was insufficient.

In Comparative Examples 199 to 210, the wafer was subjected to the same surface treatment, and evaluated in the same manner as in Comparative Examples 1 to 12, except that a protective film-forming chemical containing trimethylmethoxysilane [(CH₃)₃Si—OCH₃] in place of trimethylhexoxysilane was used. The water repellency imparting effect was not observed with the protective film-forming chemical that did not contain sulfonic acid, or with the protective film-forming chemical that contained acetic acid [CH₃C(═O)OH] instead of methanesulfonic acid, even with a change made to the alkoxy group of the alkoxysilane.

TABLE 2

| | Protective film-forming chemical | | | | | | | | | |
|---|---|---|---|---|---|---|---|---|---|---|
| | Starting raw materials | | | | | | Evaluation results | | | |
| | | | | | Diluting solvent | | | Contact | Reduction | |
| | | | | | | Alcohol | | angle | of contact | |
| | Alkoxysilane | | Acid | | | concentration | Initial | after | angle after | Resistance |
| | | Concen- | | Concen- | | in diluting | contact | surface | dipping in | of vinyl |
| | | tration | | tration | | slovent | angle | treatment | hot water | chloride |
| | Type | [mass %] | Type | [mass %] | Type | [mass %] | [°] | [°] | [°] | resin |
| Comparative | (CH₃)₃Si—OC₆H₁₃ | 20 | — | 0 | nHA | 100 | <10 | <10 | 0 | Acceptable |
| Example 1 | | | | | | | | | | |
| Comparative | | 5 | | | | | <10 | <10 | 0 | Acceptable |
| Example 2 | | | | | | | | | | |
| Comparative | | 0.5 | | | | | <10 | <10 | 0 | Acceptable |
| Example 3 | | | | | | | | | | |
| Comparative | (CH₃)₃Si—OC₆H₁₃ | 20 | CH₃C(═O)OH | 10 | | | <10 | <10 | 0 | Acceptable |
| Example 4 | | | | | | | | | | |
| Comparative | | 5 | | | | | <10 | <10 | 0 | Acceptable |
| Example 5 | | | | | | | | | | |
| Comparative | | 0.5 | | | | | <10 | <10 | 0 | Acceptable |
| Example 6 | | | | | | | | | | |
| Comparative | (CH₃)₃Si—OC₆H₁₃ | 20 | CH₃C(═O)OH | 5 | | | <10 | <10 | 0 | Acceptable |
| Example 7 | | | | | | | | | | |
| Comparative | | 5 | | | | | <10 | <10 | 0 | Acceptable |
| Example 8 | | | | | | | | | | |
| Comparative | | 0.5 | | | | | <10 | <10 | 0 | Acceptable |
| Example 9 | | | | | | | | | | |
| Comparative | (CH₃)₃Si—OC₆H₁₃ | 20 | CH₃C(═O)OH | 0.1 | | | <10 | <10 | 0 | Acceptable |
| Example 10 | | | | | | | | | | |
| Comparative | | 5 | | | | | <10 | <10 | 0 | Acceptable |
| Example 11 | | | | | | | | | | |
| Comparative | | 0.5 | | | | | <10 | <10 | 0 | Acceptable |
| Example 12 | | | | | | | | | | |
| Comparative | (CH₃)Si(OCH₃)₃ | 20 | CH₃S(═O)₂OH | 10 | | | <10 | 30 | 0 | Acceptable |
| Example 13 | | | | | | | | | | |
| Comparative | | 5 | | | | | <10 | 20 | 0 | Acceptable |
| Example 14 | | | | | | | | | | |
| Comparative | | 0.5 | | | | | <10 | <10 | 0 | Acceptable |
| Example 15 | | | | | | | | | | |
| Comparative | (CH₃)Si(OCH₃)₃ | 20 | CH₃S(═O)₂OH | 5 | | | <10 | 25 | 0 | Acceptable |
| Example 16 | | | | | | | | | | |
| Comparative | | 5 | | | | | <10 | 15 | 0 | Acceptable |
| Example 17 | | | | | | | | | | |
| Comparative | | 0.5 | | | | | <10 | <10 | 0 | Acceptable |
| Example 18 | | | | | | | | | | |
| Comparative | (CH₃)Si(OCH₃)₃ | 20 | CH₃S(═O)₂OH | 0.1 | | | <10 | <10 | 0 | Acceptable |
| Example 19 | | | | | | | | | | |
| Comparative | | 5 | | | | | <10 | <10 | 0 | Acceptable |
| Example 20 | | | | | | | | | | |
| Comparative | | 0.5 | | | | | <10 | <10 | 0 | Acceptable |
| Example 21 | | | | | | | | | | |
| Comparative | (CH₃)₃Si—OC₆H₁₃ | 20 | — | 0 | nBA | 100 | <10 | <10 | 0 | Acceptable |
| Example 22 | | | | | | | | | | |
| Comparative | | 5 | | | | | <10 | <10 | 0 | Acceptable |
| Example 23 | | | | | | | | | | |
| Comparative | | 0.5 | | | | | <10 | <10 | 0 | Acceptable |
| Example 24 | | | | | | | | | | |
| Comparative | (CH₃)₃Si—OC₆H₁₃ | 20 | CH₃C(═O)OH | 10 | | | <10 | <10 | 0 | Acceptable |
| Example 25 | | | | | | | | | | |
| Comparative | | 5 | | | | | <10 | <10 | 0 | Acceptable |
| Example 26 | | | | | | | | | | |
| Comparative | | 0.5 | | | | | <10 | <10 | 0 | Acceptable |
| Example 27 | | | | | | | | | | |
| Comparative | (CH₃)₃Si—OC₆H₁₃ | 20 | CH₃C(═O)OH | 5 | | | <10 | <10 | 0 | Acceptable |
| Example 28 | | | | | | | | | | |
| Comparative | | 5 | | | | | <10 | <10 | 0 | Acceptable |
| Example 29 | | | | | | | | | | |
| Comparative | | 0.5 | | | | | <10 | <10 | 0 | Acceptable |
| Example 30 | | | | | | | | | | |
| Comparative | (CH₃)₃Si—OC₆H₁₃ | 20 | CH₃C(═O)OH | 0.1 | | | <10 | <10 | 0 | Acceptable |
| Example 31 | | | | | | | | | | |
| Comparative | | 5 | | | | | <10 | <10 | 0 | Acceptable |
| Example 32 | | | | | | | | | | |
| Comparative | | 0.5 | | | | | <10 | <10 | 0 | Acceptable |
| Example 33 | | | | | | | | | | |
| Comparative | (CH₃)Si(OCH₃)₃ | 20 | CH₃S(═O)₂OH | 10 | | | <10 | 30 | 0 | Acceptable |
| Example 34 | | | | | | | | | | |
| Comparative | | 5 | | | | | <10 | 20 | 0 | Acceptable |
| Example 35 | | | | | | | | | | |
| Comparative | | 0.5 | | | | | <10 | <10 | 0 | Acceptable |
| Example 36 | | | | | | | | | | |
| Comparative | (CH₃)Si(OCH₃)₃ | 20 | CH₃S(═O)₂OH | 5 | | | <10 | 25 | 0 | Acceptable |
| Example 37 | | | | | | | | | | |
| Comparative | | 5 | | | | | <10 | 15 | 0 | Acceptable |
| Example 38 | | | | | | | | | | |
| Comparative | | 0.5 | | | | | <10 | <10 | 0 | Acceptable |
| Example 39 | | | | | | | | | | |
| Comparative | (CH₃)Si(OCH₃)₃ | 20 | CH₃S(═O)₂OH | 0.1 | | | <10 | <10 | 0 | Acceptable |
| Example 40 | | | | | | | | | | |
| Comparative | | 5 | | | | | <10 | <10 | 0 | Acceptable |
| Example 41 | | | | | | | | | | |
| Comparative | | 0.5 | | | | | <10 | <10 | 0 | Acceptable |
| Example 42 | | | | | | | | | | |

TABLE 3

| | Protective film-forming chemical | | | | | | | | | |
|---|---|---|---|---|---|---|---|---|---|---|
| | Starting raw materials | | | | | | Evaluation results | | | |
| | | | | | Diluting solvent | | | Contact | Reduction | |
| | | | | | | Alcohol | | angle | of contact | |
| | Alkoxysilane | | Acid | | | concentration | Initial | after | angle after | Resistance |
| | | Concen- | | Concen- | | in diluting | contact | surface | dipping in | of vinyl |
| | | tration | | tration | | slovent | angle | treatment | hot water | chloride |
| | Type | [mass %] | Type | [mass %] | Type | [mass %] | [°] | [°] | [°] | resin |
| Comparative | (CH₃)₃Si—OC₆H₁₃ | 20 | — | 0 | nPA | 100 | <10 | <10 | 0 | Acceptable |
| Example 43 | | | | | | | | | | |
| Comparative | | 5 | | | | | <10 | <10 | 0 | Acceptable |
| Example 44 | | | | | | | | | | |
| Comparative | | 0.5 | | | | | <10 | <10 | 0 | Acceptable |
| Example 45 | | | | | | | | | | |
| Comparative | (CH₃)₃Si—OC₆H₁₃ | 20 | CH₃C(═O)OH | 10 | | | <10 | <10 | 0 | Acceptable |
| Example 46 | | | | | | | | | | |
| Comparative | | 5 | | | | | <10 | <10 | 0 | Acceptable |
| Example 47 | | | | | | | | | | |
| Comparative | | 0.5 | | | | | <10 | <10 | 0 | Acceptable |
| Example 48 | | | | | | | | | | |
| Comparative | (CH₃)₃Si—OC₆H₁₃ | 20 | CH₃C(═O)OH | 5 | | | <10 | <10 | 0 | Acceptable |
| Example 49 | | | | | | | | | | |
| Comparative | | 5 | | | | | <10 | <10 | 0 | Acceptable |
| Example 50 | | | | | | | | | | |
| Comparative | | 0.5 | | | | | <10 | <10 | 0 | Acceptable |
| Example 51 | | | | | | | | | | |
| Comparative | (CH₃)₃Si—OC₆H₁₃ | 20 | CH₃C(═O)OH | 0.1 | | | <10 | <10 | 0 | Acceptable |
| Example 52 | | | | | | | | | | |
| Comparative | | 5 | | | | | <10 | <10 | 0 | Acceptable |
| Example 53 | | | | | | | | | | |
| Comparative | | 0.5 | | | | | <10 | <10 | 0 | Acceptable |
| Example 54 | | | | | | | | | | |
| Comparative | (CH₃)Si(OCH₃)₃ | 20 | CH₃S(═O)₂OH | 10 | | | <10 | 30 | 0 | Acceptable |
| Example 55 | | | | | | | | | | |
| Comparative | | 5 | | | | | <10 | 20 | 0 | Acceptable |
| Example 56 | | | | | | | | | | |
| Comparative | | 0.5 | | | | | <10 | <10 | 0 | Acceptable |
| Example 57 | | | | | | | | | | |
| Comparative | (CH₃)Si(OCH₃)₃ | 20 | CH₃S(═O)₂OH | 5 | | | <10 | 25 | 0 | Acceptable |
| Example 58 | | | | | | | | | | |
| Comparative | | 5 | | | | | <10 | 15 | 0 | Acceptable |
| Example 59 | | | | | | | | | | |
| Comparative | | 0.5 | | | | | <10 | <10 | 0 | Acceptable |
| Example 60 | | | | | | | | | | |
| Comparative | (CH₃)Si(OCH₃)₃ | 20 | CH₃S(═O)₂OH | 0.1 | | | <10 | <10 | 0 | Acceptable |
| Example 61 | | | | | | | | | | |
| Comparative | | 5 | | | | | <10 | <10 | 0 | Acceptable |
| Example 62 | | | | | | | | | | |
| Comparative | | 0.5 | | | | | <10 | <10 | 0 | Acceptable |
| Example 63 | | | | | | | | | | |
| Comparative | (CH₃)₃Si—OC₆H₁₃ | 20 | — | 0 | EA | 100 | <10 | <10 | 0 | Acceptable |
| Example 64 | | | | | | | | | | |
| Comparative | | 5 | | | | | <10 | <10 | 0 | Acceptable |
| Example 65 | | | | | | | | | | |
| Comparative | | 0.5 | | | | | <10 | <10 | 0 | Acceptable |
| Example 66 | | | | | | | | | | |
| Comparative | (CH₃)₃Si—OC₆H₁₃ | 20 | CH₃C(═O)OH | 10 | | | <10 | <10 | 0 | Acceptable |
| Example 67 | | | | | | | | | | |
| Comparative | | 5 | | | | | <10 | <10 | 0 | Acceptable |
| Example 68 | | | | | | | | | | |
| Comparative | | 0.5 | | | | | <10 | <10 | 0 | Acceptable |
| Example 69 | | | | | | | | | | |
| Comparative | (CH₃)₃Si—OC₆H₁₃ | 20 | CH₃C(═O)OH | 5 | | | <10 | <10 | 0 | Acceptable |
| Example 70 | | | | | | | | | | |
| Comparative | | 5 | | | | | <10 | <10 | 0 | Acceptable |
| Example 71 | | | | | | | | | | |
| Comparative | | 0.5 | | | | | <10 | <10 | 0 | Acceptable |
| Example 72 | | | | | | | | | | |
| Comparative | (CH₃)₃Si—OC₆H₁₃ | 20 | CH₃C(═O)OH | 0.1 | | | <10 | <10 | 0 | Acceptable |
| Example 73 | | | | | | | | | | |
| Comparative | | 5 | | | | | <10 | <10 | 0 | Acceptable |
| Example 74 | | | | | | | | | | |
| Comparative | | 0.5 | | | | | <10 | <10 | 0 | Acceptable |
| Example 75 | | | | | | | | | | |
| Comparative | (CH₃)Si(OCH₃)₃ | 20 | CH₃S(═O)₂OH | 10 | | | <10 | 30 | 0 | Acceptable |
| Example 76 | | | | | | | | | | |
| Comparative | | 5 | | | | | <10 | 20 | 0 | Acceptable |
| Example 77 | | | | | | | | | | |
| Comparative | | 0.5 | | | | | <10 | <10 | 0 | Acceptable |
| Example 78 | | | | | | | | | | |
| Comparative | (CH₃)Si(OCH₃)₃ | 20 | CH₃S(═O)₂OH | 5 | | | <10 | 25 | 0 | Acceptable |
| Example 79 | | | | | | | | | | |
| Comparative | | 5 | | | | | <10 | 15 | 0 | Acceptable |
| Example 80 | | | | | | | | | | |
| Comparative | | 0.5 | | | | | <10 | <10 | 0 | Acceptable |
| Example 81 | | | | | | | | | | |
| Comparative | (CH₃)Si(OCH₃)₃ | 20 | CH₃S(═O)₂OH | 0.1 | | | <10 | <10 | 0 | Acceptable |
| Example 82 | | | | | | | | | | |
| Comparative | | 5 | | | | | <10 | <10 | 0 | Acceptable |
| Example 83 | | | | | | | | | | |
| Comparative | | 0.5 | | | | | <10 | <10 | 0 | Acceptable |
| Example 84 | | | | | | | | | | |

TABLE 4

| | Protective film-forming chemical | | | | | | | | | |
|---|---|---|---|---|---|---|---|---|---|---|
| | Starting raw materials | | | | | | | | | |
| | | | | | Diluting solvent | | | | | |
| | | | | | | Alcohol | Evaluation results | | | |
| | Alkoxysilane | | Acid | | | concentration in | | Contact angle | Reduction of contact | |
| | | Concentraion | | Concentration | | diluting slovent | Initial contact | after surface | angle after dipping | Resistance of vinyl |
| | Type | [mass %] | Type | [mass %] | Type | [mass %] | angle [°] | treatment [°] | in hot water [°] | chloride resin |
| Comparative | (CH₃)₃Si—OC₆H₁₃ | 20 | — | 0 | nPA/PGMEA-95 | 95 | <10 | <10 | 0 | Acceptable |
| Example 85 | | | | | | | | | | |
| Comparative | | 5 | | | | | <10 | <10 | 0 | Acceptable |
| Example 86 | | | | | | | | | | |
| Comparative | | 0.5 | | | | | <10 | <10 | 0 | Acceptable |
| Example 87 | | | | | | | | | | |
| Comparative | (CH₃)₃Si—OC₆H₁₃ | 20 | CH₃C(═O)OH | 10 | | | <10 | <10 | 0 | Acceptable |
| Example 88 | | | | | | | | | | |
| Comparative | | 5 | | | | | <10 | <10 | 0 | Acceptable |
| Example 89 | | | | | | | | | | |
| Comparative | | 0.5 | | | | | <10 | <10 | 0 | Acceptable |
| Example 90 | | | | | | | | | | |
| Comparative | (CH₃)₃Si—OC₆H₁₃ | 20 | CH₃C(═O)OH | 5 | | | <10 | <10 | 0 | Acceptable |
| Example 91 | | | | | | | | | | |
| Comparative | | 5 | | | | | <10 | <10 | 0 | Acceptable |
| Example 92 | | | | | | | | | | |
| Comparative | | 0.5 | | | | | <10 | <10 | 0 | Acceptable |
| Example 93 | | | | | | | | | | |
| Comparative | (CH₃)₃Si—OC₆H₁₃ | 20 | CH₃C(═O)OH | 0.1 | | | <10 | <10 | 0 | Acceptable |
| Example 94 | | | | | | | | | | |
| Comparative | | 5 | | | | | <10 | <10 | 0 | Acceptable |
| Example 95 | | | | | | | | | | |
| Comparative | | 0.5 | | | | | <10 | <10 | 0 | Acceptable |
| Example 96 | | | | | | | | | | |
| Comparative | (CH₃)Si(OCH₃)₃ | 20 | CH₃S(═O)₂OH | 10 | | | <10 | 30 | 0 | Acceptable |
| Example 97 | | | | | | | | | | |
| Comparative | | 5 | | | | | <10 | 20 | 0 | Acceptable |
| Example 98 | | | | | | | | | | |
| Comparative | | 0.5 | | | | | <10 | <10 | 0 | Acceptable |
| Example 99 | | | | | | | | | | |
| Comparative | (CH₃)Si(OCH₃)₃ | 20 | CH₃S(═O)₂OH | 5 | | | <10 | 25 | 0 | Acceptable |
| Example 100 | | | | | | | | | | |
| Comparative | | 5 | | | | | <10 | 15 | 0 | Acceptable |
| Example 101 | | | | | | | | | | |
| Comparative | | 0.5 | | | | | <10 | <10 | 0 | Acceptable |
| Example 102 | | | | | | | | | | |
| Comparative | (CH₃)Si(OCH₃)₃ | 20 | CH₃S(═O)₂OH | 0.1 | | | <10 | <10 | 0 | Acceptable |
| Example 103 | | | | | | | | | | |
| Comparative | | 5 | | | | | <10 | <10 | 0 | Acceptable |
| Example 104 | | | | | | | | | | |
| Comparative | | 0.5 | | | | | <10 | <10 | 0 | Acceptable |
| Example 105 | | | | | | | | | | |
| Comparative | (CH₃)₃Si—OC₆H₁₃ | 20 | — | 0 | iPA | 100 | <10 | <10 | 0 | Acceptable |
| Example 106 | | | | | | | | | | |
| Comparative | | 5 | | | | | <10 | <10 | 0 | Acceptable |
| Example 107 | | | | | | | | | | |
| Comparative | | 0.5 | | | | | <10 | <10 | 0 | Acceptable |
| Example 108 | | | | | | | | | | |
| Comparative | (CH₃)₃Si—OC₆H₁₃ | 20 | CH₃C(═O)OH | 10 | | | <10 | <10 | 0 | Acceptable |
| Example 109 | | | | | | | | | | |
| Comparative | | 5 | | | | | <10 | <10 | 0 | Acceptable |
| Example 110 | | | | | | | | | | |
| Comparative | | 0.5 | | | | | <10 | <10 | 0 | Acceptable |
| Example 111 | | | | | | | | | | |
| Comparative | (CH₃)₃Si—OC₆H₁₃ | 20 | CH₃C(═O)OH | 5 | | | <10 | <10 | 0 | Acceptable |
| Example 112 | | | | | | | | | | |
| Comparative | | 5 | | | | | <10 | <10 | 0 | Acceptable |
| Example 113 | | | | | | | | | | |
| Comparative | | 0.5 | | | | | <10 | <10 | 0 | Acceptable |
| Example 114 | | | | | | | | | | |
| Comparative | (CH₃)₃Si—OC₆H₁₃ | 20 | CH₃C(═O)OH | 0.1 | | | <10 | <10 | 0 | Acceptable |
| Example 115 | | | | | | | | | | |
| Comparative | | 5 | | | | | <10 | <10 | 0 | Acceptable |
| Example 116 | | | | | | | | | | |
| Comparative | | 0.5 | | | | | <10 | <10 | 0 | Acceptable |
| Example 117 | | | | | | | | | | |
| Comparative | (CH₃)Si(OCH₃)₃ | 20 | CH₃S(═O)₂OH | 10 | | | <10 | 25 | 0 | Acceptable |
| Example 118 | | | | | | | | | | |
| Comparative | | 5 | | | | | <10 | 15 | 0 | Acceptable |
| Example 119 | | | | | | | | | | |
| Comparative | | 0.5 | | | | | <10 | <10 | 0 | Acceptable |
| Example 120 | | | | | | | | | | |
| Comparative | (CH₃)Si(OCH₃)₃ | 20 | CH₃S(═O)₂OH | 5 | | | <10 | 20 | 0 | Acceptable |
| Example 121 | | | | | | | | | | |
| Comparative | | 5 | | | | | <10 | 12 | 0 | Acceptable |
| Example 122 | | | | | | | | | | |
| Comparative | | 0.5 | | | | | <10 | <10 | 0 | Acceptable |
| Example 123 | | | | | | | | | | |
| Comparative | (CH₃)Si(OCH₃)₃ | 20 | CH₃S(═O)₂OH | 0.1 | | | <10 | <10 | 0 | Acceptable |
| Example 124 | | | | | | | | | | |
| Comparative | | 5 | | | | | <10 | <10 | 0 | Acceptable |
| Example 125 | | | | | | | | | | |
| Comparative | | 0.5 | | | | | <10 | <10 | 0 | Acceptable |
| Example 126 | | | | | | | | | | |

TABLE 5

| | Protective film-forming chemical | | | | | | | | | |
|---|---|---|---|---|---|---|---|---|---|---|
| | Starting raw materials | | | | | | | | | |
| | | | | | Diluting solvent | | | | | |
| | | | | | | Alcohol | Evaluation results | | | |
| | | | | | | concentra- | | Contact | Reduction | |
| | | | Acid | | | tion | | angle | of contact | |
| | | | | Con- | | in | Initial | after | angle after | Resistance |
| | Alkoxysilane | | | centra- | | diluting | contact | surface | dipping in | of vinyl |
| | | Concentraion | | tion | | slovent | angle | treatment | hot water | chloride |
| | Type | [mass %] | Type | [mass %] | Type | [mass %] | [°] | [°] | [°] | resin |
| Comparative | (CH₃)₃Si—OC₆H₁₃ | 20 | — | 0 | iBA | 100 | <10 | <10 | 0 | Acceptable |
| Example 127 | | | | | | | | | | |
| Comparative | | 5 | | | | | <10 | <10 | 0 | Acceptable |
| Example 128 | | | | | | | | | | |
| Comparative | | 0.5 | | | | | <10 | <10 | 0 | Acceptable |
| Example 129 | | | | | | | | | | |
| Comparative | (CH₃)₃Si—OC₆H₁₃ | 20 | CH₃C(═O)OH | 10 | | | <10 | <10 | 0 | Acceptable |
| Example 130 | | | | | | | | | | |
| Comparative | | 5 | | | | | <10 | <10 | 0 | Acceptable |
| Example 131 | | | | | | | | | | |
| Comparative | | 0.5 | | | | | <10 | <10 | 0 | Acceptable |
| Example 132 | | | | | | | | | | |
| Comparative | (CH₃)₃Si—OC₆H₁₃ | 20 | CH₃C(═O)OH | 5 | | | <10 | <10 | 0 | Acceptable |
| Example 133 | | | | | | | | | | |
| Comparative | | 5 | | | | | <10 | <10 | 0 | Acceptable |
| Example 134 | | | | | | | | | | |
| Comparative | | 0.5 | | | | | <10 | <10 | 0 | Acceptable |
| Example 135 | | | | | | | | | | |
| Comparative | (CH₃)₃Si—OC₆H₁₃ | 20 | CH₃C(═O)OH | 0.1 | | | <10 | <10 | 0 | Acceptable |
| Example 136 | | | | | | | | | | |
| Comparative | | 5 | | | | | <10 | <10 | 0 | Acceptable |
| Example 137 | | | | | | | | | | |
| Comparative | | 0.5 | | | | | <10 | <10 | 0 | Acceptable |
| Example 138 | | | | | | | | | | |
| Comparative | (CH₃)Si(OCH₃)₃ | 20 | CH₃S(═O)₂OH | 10 | | | <10 | 25 | 0 | Acceptable |
| Example 139 | | | | | | | | | | |
| Comparative | | 5 | | | | | <10 | 15 | 0 | Acceptable |
| Example 140 | | | | | | | | | | |
| Comparative | | 0.5 | | | | | <10 | <10 | 0 | Acceptable |
| Example 141 | | | | | | | | | | |
| Comparative | (CH₃)Si(OCH₃)₃ | 20 | CH₃S(═O)₂OH | 5 | | | <10 | 20 | 0 | Acceptable |
| Example 142 | | | | | | | | | | |
| Comparative | | 5 | | | | | <10 | 12 | 0 | Acceptable |
| Example 143 | | | | | | | | | | |
| Comparative | | 0.5 | | | | | <10 | <10 | 0 | Acceptable |
| Example 144 | | | | | | | | | | |
| Comparative | (CH₃)Si(OCH₃)₃ | 20 | CH₃S(═O)₂OH | 0.1 | | | <10 | <10 | 0 | Acceptable |
| Example 145 | | | | | | | | | | |
| Comparative | | 5 | | | | | <10 | <10 | 0 | Acceptable |
| Example 146 | | | | | | | | | | |
| Comparative | | 0.5 | | | | | <10 | <10 | 0 | Acceptable |
| Example 147 | | | | | | | | | | |
| Comparative | (CH₃)₃Si—OC₆H₁₃ | 20 | — | 0 | 2BA | 100 | <10 | <10 | 0 | Acceptable |
| Example 148 | | | | | | | | | | |
| Comparative | | 5 | | | | | <10 | <10 | 0 | Acceptable |
| Example 149 | | | | | | | | | | |
| Comparative | | 0.5 | | | | | <10 | <10 | 0 | Acceptable |
| Example 150 | | | | | | | | | | |
| Comparative | (CH₃)₃Si—OC₆H₁₃ | 20 | CH₃C(═O)OH | 10 | | | <10 | <10 | 0 | Acceptable |
| Example 151 | | | | | | | | | | |
| Comparative | | 5 | | | | | <10 | <10 | 0 | Acceptable |
| Example 152 | | | | | | | | | | |
| Comparative | | 0.5 | | | | | <10 | <10 | 0 | Acceptable |
| Example 153 | | | | | | | | | | |
| Comparative | (CH₃)₃Si—OC₆H₁₃ | 20 | CH₃C(═O)OH | 5 | | | <10 | <10 | 0 | Acceptable |
| Example 154 | | | | | | | | | | |
| Comparative | | 5 | | | | | <10 | <10 | 0 | Acceptable |
| Example 155 | | | | | | | | | | |
| Comparative | | 0.5 | | | | | <10 | <10 | 0 | Acceptable |
| Example 156 | | | | | | | | | | |
| Comparative | (CH₃)₃Si—OC₆H₁₃ | 20 | CH₃C(═O)OH | 0.1 | | | <10 | <10 | 0 | Acceptable |
| Example 157 | | | | | | | | | | |
| Comparative | | 5 | | | | | <10 | <10 | 0 | Acceptable |
| Example 158 | | | | | | | | | | |
| Comparative | | 0.5 | | | | | <10 | <10 | 0 | Acceptable |
| Example 159 | | | | | | | | | | |
| Comparative | (CH₃)Si(OCH₃)₃ | 20 | CH₃S(═O)₂OH | 10 | | | <10 | <10 | 0 | Acceptable |
| Example 160 | | | | | | | | | | |
| Comparative | | 5 | | | | | <10 | <10 | 0 | Acceptable |
| Example 161 | | | | | | | | | | |
| Comparative | | 0.5 | | | | | <10 | <10 | 0 | Acceptable |
| Example 162 | | | | | | | | | | |
| Comparative | (CH₃)Si(OCH₃)₃ | 20 | CH₃S(═O)₂OH | 5 | | | <10 | <10 | 0 | Acceptable |
| Example 163 | | | | | | | | | | |
| Comparative | | 5 | | | | | <10 | <10 | 0 | Acceptable |
| Example 164 | | | | | | | | | | |
| Comparative | | 0.5 | | | | | <10 | <10 | 0 | Acceptable |
| Example 165 | | | | | | | | | | |
| Comparative | (CH₃)Si(OCH₃)₃ | 20 | CH₃S(═O)₂OH | 0.1 | | | <10 | <10 | 0 | Acceptable |
| Example 166 | | | | | | | | | | |
| Comparative | | 5 | | | | | <10 | <10 | 0 | Acceptable |
| Example 167 | | | | | | | | | | |
| Comparative | | 0.5 | | | | | <10 | <10 | 0 | Acceptable |
| Example 168 | | | | | | | | | | |

TABLE 6

| | Protective film-forming chemical | | | | | | | | | |
|---|---|---|---|---|---|---|---|---|---|---|
| | Starting raw materials | | | | | | Evaluation results | | | |
| | | | | | | Alcohol | | | Reduction of | |
| | Alkoxysilane | | Acid | | | concentration | | Contact angle | contact angle | |
| | | Concentraion | | Concentration | | in diluting solvent | Initial contact | after surface | after dipping in | Resistance of |
| | Type | [mass %] | Type | [mass %] | Type | [mass %] | angle [°] | treatment [°] | hot water [°] | vinyl chloride resin |
| Comparative | (CH₃)₃Si—OC₆H₁₃ | 20 | — | 0 | tBA | 100 | <10 | <10 | 0 | Acceptable |
| Example 169 | | | | | | | | | | |
| Comparative | | 5 | | | | | <10 | <10 | 0 | Acceptable |
| Example 170 | | | | | | | | | | |
| Comparative | | 0.5 | | | | | <10 | <10 | 0 | Acceptable |
| Example 171 | | | | | | | | | | |
| Comparative | (CH₃)₃Si—OC₆H₁₃ | 20 | CH₃C(═O)OH | 10 | | | <10 | <10 | 0 | Acceptable |
| Example 172 | | | | | | | | | | |
| Comparative | | 5 | | | | | <10 | <10 | 0 | Acceptable |
| Example 173 | | | | | | | | | | |
| Comparative | | 0.5 | | | | | <10 | <10 | 0 | Acceptable |
| Example 174 | | | | | | | | | | |
| Comparative | (CH₃)₃Si—OC₆H₁₃ | 20 | CH₃C(═O)OH | 5 | | | <10 | <10 | 0 | Acceptable |
| Example 175 | | | | | | | | | | |
| Comparative | | 5 | | | | | <10 | <10 | 0 | Acceptable |
| Example 176 | | | | | | | | | | |
| Comparative | | 0.5 | | | | | <10 | <10 | 0 | Acceptable |
| Example 177 | | | | | | | | | | |
| Comparative | (CH₃)₃Si—OC₆H₁₃ | 20 | CH₃C(═O)OH | 0.1 | | | <10 | <10 | 0 | Acceptable |
| Example 178 | | | | | | | | | | |
| Comparative | | 5 | | | | | <10 | <10 | 0 | Acceptable |
| Example 179 | | | | | | | | | | |
| Comparative | | 0.5 | | | | | <10 | <10 | 0 | Acceptable |
| Example 180 | | | | | | | | | | |
| Comparative | (CH₃)Si(OCH₃)₃ | 20 | CH₃S(═O)₂OH | 10 | | | <10 | <10 | 0 | Acceptable |
| Example 181 | | | | | | | | | | |
| Comparative | | 5 | | | | | <10 | <10 | 0 | Acceptable |
| Example 182 | | | | | | | | | | |
| Comparative | | 0.5 | | | | | <10 | <10 | 0 | Acceptable |
| Example 183 | | | | | | | | | | |
| Comparative | (CH₃)Si(OCH₃)₃ | 20 | CH₃S(═O)₂OH | 5 | | | <10 | <10 | 0 | Acceptable |
| Example 184 | | | | | | | | | | |
| Comparative | | 5 | | | | | <10 | <10 | 0 | Acceptable |
| Example 185 | | | | | | | | | | |
| Comparative | | 0.5 | | | | | <10 | <10 | 0 | Acceptable |
| Example 186 | | | | | | | | | | |
| Comparative | (CH₃)Si(OCH₃)₃ | 20 | CH₃S(═O)₂OH | 0.1 | | | <10 | <10 | 0 | Acceptable |
| Example 187 | | | | | | | | | | |
| Comparative | | 5 | | | | | <10 | <10 | 0 | Acceptable |
| Example 188 | | | | | | | | | | |
| Comparative | | 0.5 | | | | | <10 | <10 | 0 | Acceptable |
| Example 189 | | | | | | | | | | |
| Comparative | (CH₃)₃Si—OC₆H₁₃ | 20 | CH₃S(═O)₂OH | 10 | nPA/PGMEA-50 | 50 | <10 | 78 | 0 | Unacceptable |
| Example 190 | | | | | | | | | | |
| Comparative | | 5 | | | | | <10 | 70 | 0 | Unacceptable |
| Example 191 | | | | | | | | | | |
| Comparative | | 0.5 | | | | | <10 | 62 | 0 | Unacceptable |
| Example 192 | | | | | | | | | | |
| Comparative | (CH₃)₃Si—OC₆H₁₃ | 20 | CH₃S(═O)₂OH | 5 | | | <10 | 73 | 0 | Unacceptable |
| Example 193 | | | | | | | | | | |
| Comparative | | 5 | | | | | <10 | 65 | 0 | Unacceptable |
| Example 194 | | | | | | | | | | |
| Comparative | | 0.5 | | | | | <10 | 55 | 0 | Unacceptable |
| Example 195 | | | | | | | | | | |
| Comparative | (CH₃)₃Si—OC₆H₁₃ | 20 | CH₃S(═O)₂OH | 0.1 | | | <10 | 64 | 0 | Unacceptable |
| Example 196 | | | | | | | | | | |
| Comparative | | 5 | | | | | <10 | 55 | 0 | Unacceptable |
| Example 197 | | | | | | | | | | |
| Comparative | | 0.5 | | | | | <10 | 50 | 0 | Unacceptable |
| Example 198 | | | | | | | | | | |
| Comparative | (CH₃)₃Si—OCH₃ | 20 | — | 0 | nHA | 100 | <10 | <10 | 0 | Acceptable |
| Example 199 | | | | | | | | | | |
| Comparative | | 5 | | | | | <10 | <10 | 0 | Acceptable |
| Example 200 | | | | | | | | | | |
| Comparative | | 0.5 | | | | | <10 | <10 | 0 | Acceptable |
| Example 201 | | | | | | | | | | |
| Comparative | (CH₃)₃Si—OCH₃ | 20 | CH₃C(═O)OH | 10 | | | <10 | <10 | 0 | Acceptable |
| Example 202 | | | | | | | | | | |
| Comparative | | 5 | | | | | <10 | <10 | 0 | Acceptable |
| Example 203 | | | | | | | | | | |
| Comparative | | 0.5 | | | | | <10 | <10 | 0 | Acceptable |
| Example 204 | | | | | | | | | | |
| Comparative | (CH₃)₃Si—OCH₃ | 20 | CH₃C(═O)OH | 5 | | | <10 | <10 | 0 | Acceptable |
| Example 205 | | | | | | | | | | |
| Comparative | | 5 | | | | | <10 | <10 | 0 | Acceptable |
| Example 206 | | | | | | | | | | |
| Comparative | | 0.5 | | | | | <10 | <10 | 0 | Acceptable |
| Example 207 | | | | | | | | | | |
| Comparative | (CH₃)₃Si—OCH₃ | 20 | CH₃C(═O)OH | 0.1 | | | <10 | <10 | 0 | Acceptable |
| Example 208 | | | | | | | | | | |
| Comparative | | 5 | | | | | <10 | <10 | 0 | Acceptable |
| Example 209 | | | | | | | | | | |
| Comparative | | 0.5 | | | | | <10 | <10 | 0 | Acceptable |
| Example 210 | | | | | | | | | | |

### Examples 22 to 79

The wafer was subjected to the same surface treatment, and evaluated in the same manner as in Example 1, except that some conditions, including the types of monoalkoxysilane, sulfonic acid, and diluting solvent were varied from those used in Example 1. The results are presented in Tables 7 to 8. In the tables, “(CH₃)₃Si—OCH₃” means trimethylmethoxysilane, “(CH₃)₃Si—OC₂H₅” means trimethylethoxysilane, “(CH₃)₃Si—OCH₂CH₂CH₃” means trimethyl-n-propoxysilane, “C₈H₁₇Si(CH₃)₂—OCH₃” means octyldimethylmethoxysilane, and “(CH₃)₂Si(H)—OC₂H₅” means dimethylethoxysilane. In the tables, “CF₃S(═O)₂OH” means trifluoromethanesulfonic acid, “C₄F₉S(═O)₂OH” means nonafluorobutanesulfonic acid, and “CH₃—C₆H₄—S(═O)₂OH” means para-toluenesulfonic acid.

In all of these examples, an initial contact angle of less than 10° before surface treatment increased after the surface treatment, demonstrating the water repellency imparting effect. There was only a small reduction of contact angle, and the water repellency was desirably maintained. The vinyl chloride resin did not deteriorate even after being stored for 4 weeks at 40° C., and the resistance was desirable.

TABLE 7

| | Protective film-forming chemical | | | | | | | | | |
|---|---|---|---|---|---|---|---|---|---|---|
| | Starting raw materials | | | | | | | | | |
| | | | | | Diluting solvent | | Evaluation results | | | |
| | | | | | | Alcohol | | | Reduction of | |
| | Monoalkoxysilane | | Sulfonic acid | | | concentration in | | Contact angle | contact angle | |
| | | Concentraion | | Concentration | | diluting slovent | Initial contact | after surface | after dipping | Resistance of vinyl |
| | Type | [mass %] | Type | [mass %] | Type | [mass %] | angle [°] | treatment [°] | in hot water [°] | chloride resin |
| Example 1 | (CH₃)₃Si—OC₆H₁₃ | 20 | CH₃S(═O)₂OH | 10 | nHA | 100 | <10 | 78 | 0 | Acceptable |
| Example 22 | (CH₃)₃Si—OCH₃ | | | | | | <10 | 77 | 0 | Acceptable |
| Example 23 | (CH₃)₃Si—OC₂H₅ | | | | | | <10 | 77 | 0 | Acceptable |
| Example 24 | (CH₃)₃Si—OCH₂CH₂CH₃ | | | | | | <10 | 78 | 0 | Acceptable |
| Example 25 | C₃H₁₇Si(CH₃)₂—OCH₃ | | | | | | <10 | 65 | 0 | Acceptable |
| Example 26 | (CH₃)₂Si(H)—OC₂H₅ | | | | | | <10 | 90 | 12 | Acceptable |
| Example 1 | (CH₃)₃Si—OC₆H₁₃ | 20 | CH₃S(═O)₂OH | 10 | | | <10 | 78 | 0 | Acceptable |
| Example 27 | | | CF₃S(═O)₂OH | | | | <10 | 70 | 0 | Acceptable |
| Example 28 | | | C₄F₉S(═O)₂OH | | | | <10 | 66 | 0 | Acceptable |
| Example 29 | | | CH₃—C₆H₄—S(═O)₂OH | | | | <10 | 55 | 0 | Acceptable |
| Example 14 | (CH₃)₃Si—OC₆H₁₃ | 20 | CH₃S(═O)₂OH | 10 | nBA | 100 | <10 | 78 | 0 | Acceptable |
| Example 30 | (CH₃)₃Si—OCH₃ | | | | | | <10 | 76 | 0 | Acceptable |
| Example 31 | (CH₃)₃Si—OC₂H₅ | | | | | | <10 | 76 | 0 | Acceptable |
| Example 32 | (CH₃)₃Si—OCH₂CH₂CH₃ | | | | | | <10 | 77 | 0 | Acceptable |
| Example 33 | C₃H₁₇Si(CH₃)₂—OCH₃ | | | | | | <10 | 66 | 0 | Acceptable |
| Example 34 | (CH₃)₂Si(H)—OC₂H₅ | | | | | | <10 | 88 | 14 | Acceptable |
| Example 14 | (CH₃)₃Si—OC₆H₁₃ | 20 | CH₃S(═O)₂OH | 10 | | | <10 | 78 | 0 | Acceptable |
| Example 35 | | | CF₃S(═O)₂OH | | | | <10 | 71 | 0 | Acceptable |
| Example 36 | | | CH₃—C₆H₄—S(═O)₂OH | | | | <10 | 54 | 0 | Acceptable |
| Example 15 | (CH₃)₃Si—OC₆H₁₃ | 20 | CH₃S(═O)₂OH | 10 | nPA | 100 | <10 | 76 | 0 | Acceptable |
| Example 37 | (CH₃)₃Si—OCH₃ | | | | | | <10 | 74 | 0 | Acceptable |
| Example 38 | (CH₃)₃Si—OC₂H₅ | | | | | | <10 | 74 | 0 | Acceptable |
| Example 39 | (CH₃)₃Si—OCH₂CH₂CH₃ | | | | | | <10 | 74 | 0 | Acceptable |
| Example 40 | C₃H₁₇Si(CH₃)₂—OCH₃ | | | | | | <10 | 65 | 0 | Acceptable |
| Example 41 | (CH₃)₂Si(H)—OC₂H₅ | | | | | | <10 | 86 | 14 | Acceptable |
| Example 15 | (CH₃)₃Si—OC₆H₁₃ | 20 | CH₃S(═O)₂OH | 10 | | | <10 | 76 | 0 | Acceptable |
| Example 42 | | | CF₃S(═O)₂OH | | | | <10 | 68 | 0 | Acceptable |
| Example 43 | | | CH₃—C₆H₄—S(═O)₂OH | | | | <10 | 56 | 0 | Acceptable |
| Example 16 | (CH₃)₃Si—OC₆H₁₃ | 20 | CH₃S(═O)₂OH | 10 | EA | 100 | <10 | 75 | 0 | Acceptable |
| Example 44 | (CH₃)₃Si—OCH₃ | | | | | | <10 | 72 | 0 | Acceptable |
| Example 45 | (CH₃)₃Si—OC₂H₅ | | | | | | <10 | 72 | 0 | Acceptable |
| Example 46 | (CH₃)₃Si—OCH₂CH₂CH₃ | | | | | | <10 | 72 | 0 | Acceptable |
| Example 47 | C₃H₁₇Si(CH₃)₂—OCH₃ | | | | | | <10 | 64 | 0 | Acceptable |
| Example 48 | (CH₃)₂Si(H)—OC₂H₅ | | | | | | <10 | 86 | 12 | Acceptable |
| Example 16 | (CH₃)₃Si—OC₆H₁₃ | 20 | CH₃S(═O)₂OH | 10 | | | <10 | 75 | 0 | Acceptable |
| Example 49 | | | CF₃S(═O)₂OH | | | | <10 | 65 | 0 | Acceptable |
| Example 50 | | | CH₃—C₆H₄—S(═O)₂OH | | | | <10 | 55 | 0 | Acceptable |

TABLE 8

| | Protective film-forming chemical | | | | | | Evaluation results | | | |
|---|---|---|---|---|---|---|---|---|---|---|
| | Starting raw materials | | | | | | | Contact | Reduction | |
| | | | | | Diluting solvent | | | angle | of contact | |
| | | | | | | Alcohol | Initial | after | angle after | |
| | Monoalkoxysilane | | Sulfonic acid | | | concentration in | contact | surface | dipping in | |
| | | Concentration | | Concentration | | diluting slovent | angle | treatment | hot water | Resistance of vinyl |
| | Type | [mass %] | Type | [mass %] | Type | [mass %] | [°] | [°] | [°] | chloride resin |
| Example 17 | (CH₃)₃Si—OC₆H₁₃ | 20 | CH₃S(═O)₂OH | 10 | nPA/PGMEA-95 | 95 | <10 | 76 | 0 | Acceptable |
| Example 51 | (CH₃)₃Si—OCH₃ | | | | | | <10 | 74 | 0 | Acceptable |
| Example 52 | (CH₃)₃Si—OC₂H₅ | | | | | | <10 | 74 | 0 | Acceptable |
| Example 53 | (CH₃)₃Si—OCH₂CH₂CH₃ | | | | | | <10 | 74 | 0 | Acceptable |
| Example 54 | C₈H₁₇Si(CH₃)₂—OCH₃ | | | | | | <10 | 66 | 0 | Acceptable |
| Example 55 | (CH₃)₂Si(H)—OC₂H₅ | | | | | | <10 | 88 | 13 | Acceptable |
| Example 17 | (CH₃)₃Si—OC₆H₁₃ | 20 | CH₃S(═O)₂OH | 10 | | | <10 | 76 | 0 | Acceptable |
| Example 56 | | | CF₃S(═O)₂OH | | | | <10 | 66 | 0 | Acceptable |
| Example 57 | | | CH₃—C₆H₄—S(═O)₂OH | | | | <10 | 56 | 0 | Acceptable |
| Example 19 | (CH₃)₃Si—OC₆H₁₃ | 20 | CH₃S(═O)₂OH | 10 | iPA | 100 | <10 | 64 | 0 | Acceptable |
| Example 58 | (CH₃)₃Si—OCH₃ | | | | | | <10 | 62 | 0 | Acceptable |
| Example 59 | (CH₃)₃Si—OC₂H₅ | | | | | | <10 | 62 | 0 | Acceptable |
| Example 60 | (CH₃)₃Si—OCH₂CH₂CH₃ | | | | | | <10 | 62 | 0 | Acceptable |
| Example 61 | C₈H₁₇Si(CH₃)₂—OCH₃ | | | | | | <10 | 52 | 0 | Acceptable |
| Example 62 | (CH₃)₂Si(H)—OC₂H₅ | | | | | | <10 | 78 | 12 | Acceptable |
| Example 19 | (CH₃)₃Si—OC₆H₁₃ | 20 | CH₃S(═O)₂OH | 10 | | | <10 | 64 | 0 | Acceptable |
| Example 63 | | | CF₃S(═O)₂OH | | | | <10 | 54 | 0 | Acceptable |
| Example 64 | | | CH₃—C₆H₄—S(═O)₂OH | | | | <10 | 50 | 0 | Acceptable |
| Example 18 | (CH₃)₃Si—OC₆H₁₃ | 20 | CH₃S(═O)₂OH | 10 | IBA | 100 | <10 | 78 | 0 | Acceptable |
| Example 65 | (CH₃)₃Si—OCH₃ | | | | | | <10 | 76 | 0 | Acceptable |
| Example 66 | (CH₃)₃Si—OC₂H₅ | | | | | | <10 | 76 | 0 | Acceptable |
| Example 67 | (CH₃)₃Si—OCH₂CH₂CH₃ | | | | | | <10 | 76 | 0 | Acceptable |
| Example 68 | C₈H₁₇Si(CH₃)₂—OCH₃ | | | | | | <10 | 65 | 0 | Acceptable |
| Example 69 | (CH₃)₂Si(H)—OC₂H₅ | | | | | | <10 | 88 | 14 | Acceptable |
| Example 18 | (CH₃)₃Si—OC₆H₁₃ | 20 | CH₃S(═O)₂OH | 10 | | | <10 | 78 | 0 | Acceptable |
| Example 70 | | | CF₃S(═O)₂OH | | | | <10 | 70 | 0 | Acceptable |
| Example 71 | | | CH₃—C₆H₄—S(═O)₂OH | | | | <10 | 55 | 0 | Acceptable |
| Example 20 | (CH₃)₃Si—OC₆H₁₃ | 20 | CH₃S(═O)₂OH | 10 | 2BA | 100 | <10 | 64 | 0 | Acceptable |
| Example 72 | (CH₃)₃Si—OCH₃ | | | | | | <10 | 62 | 0 | Acceptable |
| Example 73 | (CH₃)₃Si—OC₂H₅ | | | | | | <10 | 62 | 0 | Acceptable |
| Example 74 | (CH₃)₃Si—OCH₂CH₂CH₃ | | | | | | <10 | 62 | 0 | Acceptable |
| Example 75 | C₈H₁₇Si(CH₃)₂—OCH₃ | | | | | | <10 | 52 | 0 | Acceptable |
| Example 76 | (CH₃)₂Si(H)—OC₂H₅ | | | | | | <10 | 78 | 12 | Acceptable |
| Example 20 | (CH₃)₃Si—OC₆H₁₃ | 20 | CH₃S(═O)₂OH | 10 | | | <10 | 64 | 0 | Acceptable |
| Example 77 | | | CF₃S(═O)₂OH | | | | <10 | 54 | 0 | Acceptable |
| Example 78 | | | CH₃—C₆H₄—S(═O)₂OH | | | | <10 | 50 | 0 | Acceptable |
| Example 79 | (CH₃)₃Si—OC₆H₁₃ | 30 | CH₃S(═O)₂OH | 5 | nHA | 100 | <10 | 75 | 0 | Acceptable |

The chemicals used in the foregoing Examples are examples of the water-repellent protective film-forming chemical used by the wafer cleaning method of the present invention, and can provide a desirable water repellency imparting effect after surface treatment, and readily maintain water repellency, and do not deteriorate vinyl chloride resin even when different monoalkoxysilanes and different sulfonic acids are used in different concentrations with different diluting solvents, provided that these are within the specified ranges of the present invention.

### Comparative Examples 211 to 212

As shown in Table 9, the wafer was subjected to the same surface treatment, and evaluated in the same manner as in Example 1, except that some conditions, including the type of alkoxysilane, the type and the concentration of acid, and the type of diluting solvent were varied from those used in Example 1.

Comparative Example 211 represents an example in which a protective film-forming chemical was used that contained trimethylmethoxysilane instead of trimethylhexoxysilane, and trifluoroacetic acid [CF₃C(═O)OH] instead of methanesulfonic acid. The contact angle after the surface treatment remained low at less than 10°, and the water repellency imparting effect was not observed.

Comparative Example 212 represents an example in which trimethylmethoxysilane was contained instead of trimethylhexoxysilane, and nPA/PGMEA-50 was used as the diluting solvent. The vinyl chloride resin deteriorated (swelling was observed) after being stored for 4 weeks at 40° C., and the resistance was insufficient.

TABLE 9

| | Protective film-forming chemical | | | | | | | | | |
|---|---|---|---|---|---|---|---|---|---|---|
| | Starting raw materials | | | | | | | | | |
| | | | | | Diluting solvent | | Evaluation results | | | |
| | | | | | | Alcohol | | | Reduction | |
| | | | | | | concen- | | Contact | of contact | |
| | | | Acid | | | tration | Initial | angle after | angle after | Resistance |
| | Alkoxysilane | | | Concen- | | in diluting | contact | surface | dipping in | of vinyl |
| | | Concentraion | | tration | | slovent | angle | treatment | hot water | chloride |
| | Type | [mass %] | Type | [mass %] | Type | [mass %] | [°] | [°] | [°] | resin |
| Comparative | (CH₃)₃Si—OCH₃ | 20 | — | 0 | nHA | 100 | <10 | <10 | 0 | Acceptable |
| Example 199 | | | | | | | | | | |
| Comparative | (CH₃)₃Si—OCH₃ | 20 | CF₃C(═O)OH | 10 | nHA | 100 | <10 | <10 | 0 | Acceptable |
| Example 211 | | | | | | | | | | |
| Comparative | (CH₃)Si(OCH₃)₃ | 20 | CH₃S(═O₂)OH | 10 | nHA | 100 | <10 | 30 | 0 | Acceptable |
| Example 13 | | | | | | | | | | |
| Comparative | (CH₃)₃Si—OCH₃ | 20 | CH₃S(═O)₂OH | 10 | nPA/ | 50 | <10 | 75 | 0 | Un- |
| Example 212 | | | | | PGMEA- | | | | | acceptable |
| | | | | | 50 | | | | | |

The chemicals used in the foregoing Comparative Examples are examples of a chemical that is not the water-repellent protective film-forming chemical used by the wafer cleaning method of the present invention, and cannot impart water repellency after surface treatment, and deteriorate vinyl chloride resin even when the chemicals contain different alkoxysilanes and different acids in different concentrations with different diluting solvents, so long as these are outside of the specified ranges of the present invention.

### Example 80

A protective film-forming chemical containing trifluoromethanesulfonic acid as the sulfonic acid was obtained by mixing and reacting 20 g of trimethylmethoxysilane (monoalkoxysilane), 18.8 g of trifluoromethanesulfonic acid anhydride [{CF₃S(═O)₂}₂O] (acid A), and 61.2 g of nHA (diluting solvent), as shown in Table 10. The wafer was subjected to the same surface treatment, and evaluated in the same manner as in Example 1 except that this chemical was used. An initial contact angle of less than 10° before surface treatment increased to 72° after the surface treatment, demonstrating the water repellency imparting effect. There was no reduction (0°) in contact angle, and the water repellency was desirably maintained. The vinyl chloride resin did not deteriorate even after being stored for 4 weeks at 40° C., and the resistance was desirable.

TABLE 10

| | Protective film-forming chemical | | | | | |
|---|---|---|---|---|---|---|
| | Starting raw materials | | | | Monoalkoxysilane | |
| | | | | Diluting | | Concentration |
| | Monoalkoxysilane | Silylation agent | Acid A | solvent | Type | [mass %] |
| Example 80 | (CH₃)₃Si—OCH₃ | — | [CF₃S(═O)₂]₂O | nHA | (CH₃)₃Si—OCH₃ | 20 |
| Example 81 | (CH₃)₃Si—OC₂H₅ | — | [CF₃S(═O)₂]₂O | nHA | (CH₃)₃Si—OC₂H₅ | 20 |
| Example 82 | (CH₃)₃Si—OC₆H₁₃ | — | [CF₃S(═O)₂]₂O | nHA | (CH₃)₃Si—OC₆H₁₃ | 20 |
| Example 83 | (CH₃)₃Si—OCH₃ | — | [CF₃S(═O)₂]₂O | nPA | (CH₃)₃Si—OCH₃ | 20 |
| Example 84 | (CH₃)₃Si—OC₂H₅ | — | [CF₃S(═O)₂]₂O | nPA | (CH₃)₃Si—OC₂H₅ | 20 |
| Example 85 | (CH₃)₃Si—OC₆H₁₃ | — | [CF₃S(═O)₂]₂O | nPA | (CH₃)₃Si—OC₆H₁₃ | 20 |
| Example 86 | (CH₃)₃Si—OCH₃ | — | [CH₃S(═O)₂]₂O | nHA | (CH₃)₃Si—OCH₃ | 20 |
| Example 87 | (CH₃)₃Si—OC₂H₅ | — | [CH₃S(═O)₂]₂O | nHA | (CH₃)₃Si—OC₂H₅ | 20 |
| Example 88 | (CH₃)₃Si—OC₆H₁₃ | — | [CH₃S(═O)₂]₂O | nHA | (CH₃)₃Si—OC₆H₁₃ | 20 |
| Example 89 | (CH₃)₃Si—OCH₃ | — | [CH₃S(═O)₂]₂O | nPA | (CH₃)₃Si—OCH₃ | 20 |
| Example 90 | (CH₃)₃Si—OC₂H₅ | — | [CH₃S(═O)₂]₂O | nPA | (CH₃)₃Si—OC₂H₅ | 20 |
| Example 91 | (CH₃)₃Si—OC₆H₁₃ | — | [CH₃S(═O)₂]₂O | nPA | (CH₃)₃Si—OC₆H₁₃ | 20 |
| Example 92 | — | (CH₃)₃Si—OS(═O)₂CF₃ | — | nBA | (CH₃)₃Si—OCH₂CH₂CH₂CH₃ | 20 |
| Example 93 | — | (CH₃)₃Si—OS(═O)₂CF₃ | — | nPA | (CH₃)₃Si—OCH₂CH₂CH₃ | 20 |
| Example 94 | — | (CH₃)₃Si—OS(═O)₂CF₃ | — | nHA | (CH₃)₃Si—OCH₂CH₂CH₂CH₂CH₂CH₃ | 20 |
| Example 95 | — | (CH₃)₃Si—OS(═O)₂CF₃ | — | iPA | (CH₃)₃Si—OCH(CH₃)₂ | 20 |
| Example 96 | — | (CH₃)₃Si—OS(═O)₂CH₃ | — | nBA | (CH₃)₃Si—OCH₂CH₂CH₂CH₃ | 20 |
| Example 97 | — | (CH₃)₃Si—OS(═O)₂CH₃ | — | nPA | (CH₃)₃Si—OCH₂CH₂CH₃ | 20 |
| Example 98 | — | (CH₃)₃Si—OS(═O)₂CH₃ | — | nHA | (CH₃)₃Si—OCH₂CH₂CH₂CH₂CH₂CH₃ | 20 |
| Example 99 | — | (CH₃)₃Si—OS(═O)₂CH₃ | — | iPA | (CH₃)₃Si—OCH(CH₃)₂ | 20 |

| | Protective | | | | | |
|---|---|---|---|---|---|---|
| | film-forming chemical | | Evaluation results | | | |
| | Sulfonic acid | | | Contact angle | Reduction of contact angle | |
| | | Concentration | Initial contact | after surface treatment | after dipping in hot water | Resistance of vinyl |
| | Type | [mass %] | angle [°] | [°] | [°] | chloride resin |
| Example 80 | CF₃S(═O)₂OH | 10.0 | <10 | 72 | 0 | Acceptable |
| Example 81 | | | <10 | 72 | 0 | Acceptable |
| Example 82 | | | <10 | 74 | 0 | Acceptable |
| Example 83 | CF₃S(═O)₂OH | 10.0 | <10 | 70 | 0 | Acceptable |
| Example 84 | | | <10 | 70 | 0 | Acceptable |
| Example 85 | | | <10 | 72 | 0 | Acceptable |
| Example 86 | CH₃S(═O)₂OH | 10.0 | <10 | 80 | 0 | Acceptable |
| Example 87 | | | <10 | 80 | 0 | Acceptable |
| Example 88 | | | <10 | 82 | 0 | Acceptable |
| Example 89 | CH₃S(═O)₂OH | 10.0 | <10 | 78 | 0 | Acceptable |
| Example 90 | | | <10 | 78 | 0 | Acceptable |
| Example 91 | | | <10 | 80 | 0 | Acceptable |
| Example 92 | CF₃S(═O)₂OH | 20.5 | <10 | 80 | 0 | Acceptable |
| Example 93 | CF₃S(═O)₂OH | 22.7 | <10 | 80 | 0 | Acceptable |
| Example 94 | CF₃S(═O)₂OH | 17.2 | <10 | 80 | 0 | Acceptable |
| Example 95 | CF₃S(═O)₂OH | 22.7 | <10 | 68 | 0 | Acceptable |
| Example 96 | CF₃S(═O)₂OH | 13.2 | <10 | 84 | 0 | Acceptable |
| Example 97 | CH₃S(═O)₂OH | 14.5 | <10 | 84 | 0 | Acceptable |
| Example 98 | CH₃S(═O)₂OH | 11.0 | <10 | 84 | 0 | Acceptable |
| Example 99 | CH₃S(═O)₂OH | 14.5 | <10 | 70 | 0 | Acceptable |

### Examples 81 to 91

The wafer was subjected to the surface treatment, and evaluated by varying some conditions, including the monoalkoxysilane, the acid A, and the diluting solvent used in Example 80. The results presented in Table 10. In the table, “{CH₃S(═O)₂}₂O” means methanesulfonic acid anhydride.

In all of these examples, an initial contact angle of less than 10° before surface treatment increased after the surface treatment, demonstrating the water repellency imparting effect. There was only a small reduction of contact angle, and the water repellency was desirably maintained. The vinyl chloride resin did not deteriorate even after being stored for 4 weeks at 40° C., and the resistance was desirable.

### Example 92

A protective film-forming chemical containing trimethyl-n-butoxy silane [(CH₃)₃Si—OCH₂CH₂CH₂CH₃] as the monoalkoxysilane, and trifluoromethanesulfonic acid as the sulfonic acid was obtained by mixing and reacting 33.6 g of trimethylsilyltrifluoromethanesulfonate [(CH₃)₃Si—OS(═O)₂CF₃] as a silylation agent, and 66.4 g of nBA as the diluting solvent, as shown in Table 10. The wafer was subjected to the same surface treatment, and evaluated in the same manner as in Example 1 except that this chemical was used. An initial contact angle of less than 10° before surface treatment increased to 80° after the surface treatment, demonstrating the water repellency imparting effect. There was no reduction (0°) in contact angle, and the water repellency was desirably maintained. The vinyl chloride resin did not deteriorate even after being stored for 4 weeks at 40° C., and the resistance was desirable.

### Examples 93 to 99

The wafer was subjected to the surface treatment, and evaluated by varying some conditions, including the silylation agent, and the diluting solvent used in Example 92. The results presented in Table 10. In the table, “(CH₃)₃Si—OS(═O)₂CH₃” means trimethylsilylmethanesulfonate, “(CH₃)₃Si—OCH₂CH₂CH₃” means trimethyl-n-propoxysilane, “(CH₃)₃Si—OCH₂CH₂CH₂CH₂CH₂CH₃” means trimethyl-n-hexoxy silane, and (CH₃)₃Si—OCH(CH₃)₂ means “trimethylisopropoxysilane”. In all of these examples, an initial contact angle of less than 10° before surface treatment increased after the surface treatment, demonstrating the water repellency imparting effect. There was only a small reduction of contact angle, and the water repellency was desirably maintained. The vinyl chloride resin did not deteriorate even after being stored for 4 weeks at 40° C., and the resistance was desirable.

### Comparative Example 213

A protective film-forming chemical was obtained in the same manner as in Example 1, except that 16.5 g of trimethylchlorosilane [(CH₃)₃Si—Cl] as a silylation agent, and 83.5 g of nPA as the diluting solvent were mixed and reacted to produce a protective film-forming chemical containing trimethyl-n-propoxysilane (monoalkoxysilane) and hydrogen chloride. Specifically, in this Comparative Example, a protective film-forming chemical was used that contained a non-sulfonic acid instead of the sulfonic acid. The evaluation results are presented in Table 11. The vinyl chloride resin deteriorated (discoloration was observed) after being stored for 4 weeks at 40° C., and the resistance was insufficient.

TABLE 11

| | Protective film-forming chemical | | | | |
|---|---|---|---|---|---|
| | Starting raw materials | | | | |
| | | | | Diluting | Monoalkoxysilane |
| | Monoalkoxysilane | Silylation agent | Acid A | solvent | Type |
| Comparative | — | (CH₃)₃Si—Cl | — | nPA | (CH₃)₃Si—OCH₂CH₂CH₃ |
| Example 213 | | | | | |
| Comparative | — | (CH₃)₃Si—Cl | — | nBA | (CH₃)₃Si—OCH₂CH₂CH₂CH₃ |
| Example 214 | | | | | |
| Comparative | — | (CH₃)₃Si—Cl | — | nHA | (CH₃)₃Si—OCH₂CH₂CH₂CH₂CH₂CH₃ |
| Example 215 | | | | | |
| Comparative | — | (CH₃)₃Si—Cl | — | iPA | (CH₃)₃Si—OCH(CH₃)₂ |
| Example 216 | | | | | |

| | | Evaluation results | | | |
|---|---|---|---|---|---|
| | | | Contact | Reduction | |
| Protective | | | angle | of contact | |
| film-forming chemical | | Initial | after | angle after | |
| Monoalkoxysilane | Sulfonic acid | contact | surface | dipping in | Resistance of |

| | Concentration | | Concentration | angle | treatment | hot water | vinyl chloride |
|---|---|---|---|---|---|---|---|
| | [mass %] | Type | [mass %] | [°] | [°] | [°] | resin |
| Comparative | 20 | HCl | 5.5 | <10 | 75 | 0 | Unacceptable |
| Example 213 | | | | | | | |
| Comparative | 20 | HCl | 5.0 | <10 | 75 | 0 | Unacceptable |
| Example 214 | | | | | | | |
| Comparative | 20 | HCl | 4.2 | <10 | 75 | 0 | Unacceptable |
| Example 215 | | | | | | | |
| Comparative | 20 | HCl | 5.5 | <10 | 70 | 0 | Unacceptable |
| Example 216 | | | | | | | |

### Comparative Examples 214 to 216

The wafer was subjected to the same surface treatment, and evaluated in the same manner as in Comparative Example 213, except that the type of diluting solvent was varied, as shown in Table 11. As in Comparative Example 213, the vinyl chloride resin deteriorated (discoloration was observed) after being stored for 4 weeks at 40° C., and the resistance was insufficient.

The chemicals used in the foregoing Examples are examples of the water-repellent protective film-forming chemical used by the wafer cleaning method of the present invention, and can provide a desirable water repellency imparting effect after surface treatment, and readily maintain water repellency, and do not deteriorate vinyl chloride resin even when different monoalkoxysilanes and different sulfonic acids are used in different concentrations with different diluting solvents, provided that these are within the specified ranges of the present invention.

### REFERENCE SIGNS LIST

1 Wafer
2 Fine uneven pattern on wafer surface
3 Raised portion of pattern
4 Recessed portion of pattern
5 Width of recessed portion
6 Height of raised portion
7 Width of raised portion
8 Protective film-forming chemical retained in recessed portion 4
9 Liquid retained in recessed portion 4
10 Protective film

1. A method for cleaning a wafer having a fine uneven surface pattern that at least partially contains a silicon element using a wafer cleaning device that includes a vinyl chloride resin as a liquid contacting member,
the method comprising retaining a water-repellent protective film-forming chemical in at least a recessed portion of the uneven pattern to form a water-repellent protective film on a surface of the recessed portion,
the water-repellent protective film-forming chemical comprising:
a monoalkoxysilane represented by the following general formula [1];
a sulfonic acid represented by the following general formula [2]; and
a diluting solvent,
wherein the diluting solvent contains 80 to 100 mass % of alcohol with respect to the total 100 mass % of the diluting solvent,
(R¹)ₐSi(H)₃₋ₐ(OR²) [1],
wherein R¹ each independently represent at least one selected from monovalent hydrocarbon groups having 1 to 18 carbon atoms in which hydrogen elements may partially or totally be replaced with a fluorine element, R² represents a monovalent hydrocarbon group having 1 to 18 carbon atoms in which hydrogen elements may partially or totally be replaced with a fluorine element, and a is an integer of 1 to 3,
R³—S(═O)₂OH [2],
wherein R³ represents a group selected from the group containing a monovalent hydrocarbon group having 1 to 8 carbon atoms in which hydrogen elements may partially or totally be replaced with a fluorine element, and a hydroxyl group.

2. The method for cleaning the wafer as claimed in claim 1, wherein R³ of the sulfonic acid represented by the general formula [2] is a linear alkyl group having 1 to 8 carbon atoms in which hydrogen elements may partially or totally be replaced with a fluorine element.

3. The method for cleaning the wafer as claimed in claim 1, wherein the alcohol is a primary alcohol having 1 to 8 carbon atoms.

4. The method for cleaning the wafer as claimed in claim 1, wherein the monoalkoxysilane is at least one selected from the group containing monoalkoxysilanes represented by the following general formula [3]
R⁴—Si(CH₃)₂(OR⁵) [3],
wherein R⁴ represents a monovalent hydrocarbon group having 1 to 8 carbon atoms in which hydrogen elements may partially or totally be replaced with a fluorine element, and R⁵ represents a monovalent hydrocarbon group having 1 to 8 carbon atoms.

5. The method for cleaning the wafer as claimed in claim 1, wherein a concentration of the monoalkoxysilane in the water-repellent protective film-forming chemical is 0.5 to 35 mass %.

6. The method for cleaning the wafer as claimed in claim 1, wherein a concentration of the sulfonic acid in the water-repellent protective film-forming chemical is 0.1 to 30 mass %.

7. The method for cleaning the wafer as claimed in claim 1, wherein the water-repellent protective film-forming chemical is removed from the recessed portion by being dried after the water-repellent protective film is formed on the surface of the recessed portion with the water-repellent protective film-forming chemical retained in at least the recessed portion of the uneven pattern.

8. The method for cleaning the wafer as claimed in claim 1, wherein the water-repellent protective film-forming chemical in the recessed portion is replaced with a cleaning liquid different from the chemical, and the cleaning liquid is removed from the recessed portion by being dried after the water-repellent protective film is formed on the surface of the recessed portion with the water-repellent protective film-forming chemical retained in at least the recessed portion of the uneven pattern.

9. The method for cleaning the wafer as claimed in claim 7, wherein the water-repellent protective film is removed by subjecting the dried wafer surface to at least one selected from the group containing a heat treatment, photo-irradiation, exposure to ozone, plasma irradiation, and corona discharge.

10. A water-repellent protective film-forming chemical used when cleaning a wafer having a fine uneven surface pattern that at least partially contains a silicon element using a wafer cleaning device that includes a vinyl chloride resin as a liquid contacting member,
the water-repellent protective film-forming chemical comprising:
a monoalkoxysilane represented by the following general formula [1];
a sulfonic acid represented by the following general formula [2]; and
a diluting solvent,
wherein the diluting solvent contains 80 to 100 mass % of alcohol with respect to the total 100 mass % of the diluting solvent,
(R¹)ₐSi(H)₃₋ₐ(OR²) [1],
wherein R¹ each independently represent at least one selected from monovalent hydrocarbon groups having 1 to 18 carbon atoms in which hydrogen elements may partially or totally be replaced with a fluorine element, R² represents a monovalent hydrocarbon group having 1 to 18 carbon atoms in which hydrogen elements may partially or totally be replaced with a fluorine element, and a is an integer of 1 to 3,
R³—S(═O)₂OH [2],
wherein R³ represents a group selected from the group containing a monovalent hydrocarbon group having 1 to 8 carbon atoms in which hydrogen elements may partially or totally be replaced with a fluorine element, and a hydroxyl group.

11. The water-repellent protective film-forming chemical as claimed in claim 10, wherein R³ of the sulfonic acid represented by the general formula [2] is a linear alkyl group having 1 to 8 carbon atoms in which hydrogen elements may partially or totally be replaced with a fluorine element.

12. The water-repellent protective film-forming chemical as claimed in claim 10, wherein the alcohol is a primary alcohol having 1 to 8 carbon atoms.

13. The water-repellent protective film-forming chemical as claimed in claim 10, wherein the monoalkoxysilane is at least one selected from the group containing monoalkoxysilanes represented by the following general formula [3]
R⁴—Si(CH₃)₂(OR⁵) [3],
wherein R⁴ represents a monovalent hydrocarbon group having 1 to 8 carbon atoms in which hydrogen elements may partially or totally be replaced with a fluorine element, and R⁵ represents a monovalent hydrocarbon group having 1 to 8 carbon atoms.

14. The water-repellent protective film-forming chemical as claimed in claim 10, wherein a concentration of the monoalkoxysilane in the water-repellent protective film-forming chemical is 0.5 to 35 mass %.

15. The water-repellent protective film-forming chemical as claimed in claim 10, wherein a concentration of the sulfonic acid in the water-repellent protective film-forming chemical is 0.1 to 30 mass %.

16. The method for cleaning the wafer as claimed in claim 8, wherein the water-repellent protective film is removed by subjecting the dried wafer surface to at least one selected from the group containing a heat treatment, photo-irradiation, exposure to ozone, plasma irradiation, and corona discharge.
